(19) United States
(12) Patent Application Publication
(10) Pub. No.: US 2007/0269371 A1
(43) Pub. Date: Nov. 22, 2007

(54) Antibodies with altered effector functions

(75) Inventors: Lynne A. Krummen, San Francisco, CA (US); Dorothea Reilly, San Francisco, CA (US); Stefanie Weikert, Redwood City, CA (US)

Correspondence Address: GENENTECH, INC., 1 DNA WAY, SOUTH SAN FRANCISCO, CA, 94080, US

(73) Assignee: Genentech, Inc.

(21) Appl. No.: 11/713,577

(22) Filed: Feb. 28, 2007

Related U.S. Application Data
(63) Continuation of application No. 10/934,087, filed on Sep. 3, 2004, now abandoned.
(60) Provisional application No. 60/500,622, filed on Sep. 5, 2003.

(51) Int. Cl. A61K 39/395; A61K 35/00; A61P 37/00

(52) U.S. Cl. 424/1.49; 424/133.1; 424/134.1; 424/178.1

(57) ABSTRACT

The invention provides antibodies with altered effector functions, and methods of using these antibodies in the treatment of various diseases. The invention further provides compositions, kits and articles of manufacture for practicing methods of the invention.

### RELATED APPLICATIONS

This application is a continuation application of U.S. application Ser. No. 10/934,087, filed 3 Sep. 2004, which in turn claims priority under 35 U.S.C. 119(e) to U.S. Provisional Application No. 60/500,622 filed 5 Sep. 2003, which applications are hereby incorporated by reference in their entirety.

### TECHNICAL FIELD

The present invention relates generally to the fields of molecular biology and protein technology. More specifically, the invention concerns recombinantly produced antibodies, methods of making and uses thereof.

### BACKGROUND

Recent years have seen increasing promises of using antibodies as diagnostic and therapeutic agents for various disorders and diseases. The importance of antibodies in general for diagnostic, research and therapeutic purposes is reflected in the significant amount of effort that has been expended to study, and to modify antibody sequences and structures, from those found in natural antibodies, to achieve desired characteristics. Such attempts are well established in the art. See, for example, U.S. Pat. Nos. 6,165,745; 5,854,027; WO 95/14779; WO 99/25378; Chamow et al., J. Immunol. (1994), 153:4268-4280; Merchant et al., Nature Biotech. (1998), 16:677-681; Adlersberg, Ric. Clin. Lab. (1976), 6(3):191-205. Modifications of antibody sequences, for example those of the framework, are common.

In general, however, the art recognizes that certain residues perform critical roles in conferring biochemical and functional characteristics associated with antibodies, and therefore modifications of these residues must be made with care, if at all. One such group of residues is comprised of conserved cysteine residues that form intrachain and/or interchain disulfide linkages. Conservation of these cysteines, and the apparent structural role they play, suggests that their absence or modification could lead to undesirable results. Indeed, even where attempts have been made to modify these cysteines, the thought appears to be that (i) at least a portion of the function of these cysteines must be retained in order to preserve an acceptable level of antibody integrity, function and activity; or (ii) the modification(s) can be made only in the context of antibody fragments rather than full length antibodies. See, for example, U.S. Pat. Nos. 5,892,019; 5,348,876; 5,648,237; 5,677,425; WO 92/22583; WO 99/64460; Kim et al., Mol. Immunol. (1995), 32(7):467-475. Furthermore, in situations involving absence or deletion of a genetic hinge, such as described in Brekke et al. (Nature (1993), 363:628-630), a disulfide linkage is artificially introduced to compensate for loss of disulfide linkages resulting from the absence of wild type hinge cysteines.

Certain modifications of sequences and structures of naturally occurring monoclonal antibodies can lead to clinically useful proteins with unusual functional and biochemical characteristics. Presta, L., Current Pharmaceutical Biotechnology (2002), 237-256. For example, in instances where the therapeutic aspect of an antibody does not require effector functions such as FcγR binding (and thus antibody-dependent cell-mediated cytotoxicity (ADCC) and/or phagocytosis), or in instances where effector function of a therapeutic antibody may be detrimental, it is generally deemed to be desirable to ablate or substantially reduce such effector functions. Many attempts to identify appropriate modifications that result in antibodies that exhibit the appropriate characteristics have been made. See, for e.g., Hsu et al., Transplantation (1999), 27:68(4):545-554; Carpenter et al., J. Immunol. (2000), 165:6205-6213; Xu et al., Cell. Immunol. (2000), 200:16-26; Van der Lubbe et al., Arthritis Rheum. (1993), 36(10):1375-1379; Kon et al., Lancet (1998), 352:1109-1113; Reddy et al., J. Immunol. (2000), 164:1925-1933; Duncan et al., Nature (1988), 332:563-564; Klein et al., Proc. Natl. Acad. Sci. USA (1981), 78(1):524-528; Gillies & Wesolowski, Hum. Antibod. Hybridomas (1990), 1(1):47-54; and Armour et al., Eur. J. Immunol. (1999), 29:2613-2624. One important factor that further complicates these attempts is the need to ensure that such modifications do not significantly compromise the pharmacokinetic characteristics of the modified antibody. For example, retention of substantially wild type in vivo half life or clearance is important in many clinical settings.

Monoclonal antibodies elicit four main effector functions: ADCC, phagocytosis, complement-dependent cytotoxicity (CDC), and half life/clearance rate. ADCC and phagocytosis are mediated through interaction of cell-bound monoclonal antibodies with Fc gamma receptors (FcγR), CDC by interaction of cell-bound mAbs with the series of soluble blood proteins that constitute the complement system (e.g., C1q), and for half-life by binding of free monoclonal antibody to the neonatal Fc receptor (FcRn). Presta, Current Pharmaceutical Biotechnology (2002), 237-256. Proper glycosylation of the Fc region of a monoclonal antibody (such as IgG) is thought to be important in conferring wild type effector functions. See, for e.g., Jefferis & Lund, Immunol. Lett. (2002), 82(1-2):57-65; Lisowska, Cell. Mol. Life. Sci. (2002), 59(3):445-455; Radaev & Sun, Mol. Immunol. (2002), 38(14): 1073-1083; Mimura et al., Adv. Exp. Med. Biol. (2001), 495:49-53; Rudd et al., Science (2001), 291(5512):2370-2376; Jefferis et al., Immunol. Rev. (1998), 163:59-76; Wright & Morrison, Trends Biotechnol. (1997), 15(1):26-32; Jefferis & Lund, Chem. Immunol. (1997), 65:111-128.

Despite widespread efforts, there remains a significant and serious need for improved therapeutic methods based on using antibodies that are capable of exerting the desirable biological effects, yet exhibit reduced undesirable effector function-associated side effects. The invention described herein addresses this need and provides other benefits.

All references cited herein, including patent applications and publications, are incorporated by reference in their entirety.

### DISCLOSURE OF THE INVENTION

The invention provides methods, compositions, kits and articles of manufacture for using immunoglobulins, preferably antibodies, that are produced in eukaryotic host cells and exhibit reduced capability to form disulfide linkages, said immunoglobulins preferably comprising a variant heavy chain, in particular a variant hinge region in the heavy chain. Immunoglobulins/antibodies of the invention may comprise wild type Fc region glycosylation profiles, yet possess only a subset of wild type effector functions.

Eukaryotically generated antibodies comprising variant hinge regions were discovered to be capable of effecting therapeutic alleviation/amelioration of disease in vivo. These antibodies comprise a variant hinge region wherein cysteines that are normally capable of forming inter-heavy chain disulfide linkages are rendered incapable of forming such linkages. Surprisingly, analytical characterization of these antibodies showed no significant differences in product quality compared to antibodies comprising the wild type hinge region counterpart. Furthermore, these antibodies exhibited significantly reduced or an absence altogether of binding to various Fcγ receptors (such as FcγRIII). These receptors are thought to play an important role in effecting effector functions such as antibody dependent cellular cytotoxicity (ADCC). ADCC activity of these antibodies appears to be significantly reduced. Advantageously, binding to the FcRn is not affected, and therefore these antibodies exhibit similar/comparable clearance compared to their wild type counterpart, and are moreover substantially similar to their wild type counterpart in therapeutic utility in vivo. This demonstrates a highly advantageous method of treating diseases, wherein a simple variation of a sequence in the hinge region results in therapeutic antibodies that have the necessary therapeutic functions but lack unnecessary or undesirable full length antibody-specific characteristics (i.e., where certain effector functions such as ADCC which are normally associated with full length antibodies comprising wild type Fc regions (generally necessary for retention of wild type half life in vivo) are unnecessary or deleterious). Furthermore, these antibodies can generally be produced in host cells without significant reduction in product yield, suggesting that important factors such as stability, proper folding and assembly are not negatively affected by the presence of the variation in the hinge region (and the elimination of interheavy chain disulfide linkages). Antibodies of the invention as described herein are ideal for clinical situations wherein a therapeutic antibody exerts its therapeutic function without involving unnecessary or undesirable immune system effector functions (such as ADCC and/or CDC), while its half life/clearance in vivo remains substantially similar to wild type levels.

In one aspect, an antibody of the invention lacks intermolecular disulfide linkage (for e.g., disulfide linkage between two heavy chains). In some embodiments, said inter-heavy chain disulfide linkage is between Fc regions. In another embodiment, an antibody of the invention comprises a variant heavy chain hinge region incapable of, or that participate in, intermolecular disulfide linkage. In one embodiment, said variant hinge region lacks at least one cysteine, at least two, at least three, at least four, or any integer number up to all, cysteines normally present in a wild type hinge region that are capable of forming an intermolecular (for e.g., inter-heavy chain) disulfide linkage. In general, antibodies of the invention possess substantially similar biological (such as, but not limited to, antigen binding capability) and/or physicochemical characteristics relevant for therapeutic effects as their wild type counterparts, except that antibodies of the invention substantially lack at least one, but not all, of the effector functions of the wild type counterpart antibody. For e.g., the effector functions would include those known in the art to be associated with the Fc region, such as ADCC, phagocytosis, CDC and half life/clearance. In some embodiments, an antibody of the invention has reduced, or substantially or completely lacks, ADCC activity, but comprises substantially similar FcRn binding compared to its wild type counterpart. For e.g., an antibody of the invention may exhibit cytotoxicity levels that are 50% or less, 40% or less, 30% or less, 20% or less, 10% or less, 5% or less of the cytotoxicity levels exhibited by a wild type counterpart antibody when ADCC activity is assessed under similar assay conditions. Such assays can be any known in the art, including those described herein. In some embodiments, binding of an antibody of the invention to FcγR is reduced compared to wild type. In one embodiment, binding to FcγIa receptor is decreased compared to the wild type counterpart antibody. For example, the EC50 value of an antibody of the invention can be at least 2-fold, 3-fold, 4-fold, 5-fold, 8-fold, 10-fold of the EC50 value of a wild type counterpart antibody when binding is assessed under similar assay conditions. In some embodiments, binding of an antibody of the invention to FcγIa receptor is reduced, but not completely abolished. In one embodiment, binding to FcγIIa and/or FcγIIb receptors is decreased compared to the wild type counterpart antibody. For example, the EC50 value of an antibody of the invention can be at least 2-fold, 3-fold, 4-fold, 5-fold, 8-fold, 10-fold of the EC50 value of a wild type counterpart antibody when binding is assessed under similar assay conditions. In one embodiment, binding to FcγIII is reduced compared to a wild type counterpart antibody. For example, the EC50 value of an antibody of the invention can be at least 2-fold, 3-fold, 4-fold, 5-fold, 8-fold, 10-fold of the EC50 value of a wild type counterpart antibody when binding is assessed under similar assay conditions. In one embodiment, binding to at least one of FcγIa, FcγIIa, FcγIIb and FcγIII is reduced compared to a wild type counterpart antibody. For example, the EC50 value of an antibody of the invention can be at least 2-fold, 3-fold, 4-fold, 5-fold, 8-fold, 10-fold of the EC50 value of a wild type counterpart antibody when binding is assessed under similar assay conditions. In some embodiments, an antibody of the invention has reduced, or substantially or completely lacks, CDC activity, but comprises substantially similar FcRn binding compared to its wild type counterpart. For e.g., levels of CDC activity of an antibody of the invention can be 50% or less, 40% or less, 30% or less, 20% or less, 10% or less, 5% or less of the levels exhibited by a wild type counterpart antibody when CDC activity is assessed under similar assay conditions. In some embodiments, an antibody of the invention has reduced, or substantially or completely lacks, binding to a complement protein, for e.g. C1q, but comprises substantially similar FcRn binding compared to its wild type counterpart. For example, the EC50 value of an antibody of the invention for binding to a complement protein (such as C1q) can be at least 2-fold, 3-fold, 4-fold, 5-fold, 8-fold, 10-fold of the EC50 value of a wild type counterpart antibody when binding is assessed under similar assay conditions. In some embodiments, an antibody of the invention has reduced, or substantially or completely lacks, ADCC and CDC activity, but comprises substantially similar FcRn binding compared to its wild type counterpart. In some embodiments, an antibody of the invention has reduced, or substantially or completely lacks, ADCC activity and binding to a complement protein (such as C1q), but comprises substantially similar FcRn binding compared to its wild type counterpart. In some embodiments, an antibody of the invention comprises substantially similar or identical Fc glycosylation profile as a wild type counterpart antibody.

In some embodiments, the invention provides an antibody comprising a variant hinge region of an immunoglobulin heavy chain, wherein said variant hinge region lacks (i.e., does not comprise or contain, or is free of) a cysteine residue capable of forming a disulfide linkage. In some embodiments, said disulfide linkage is intermolecular (preferably inter-heavy chain). In some embodiments of antibodies wherein two or more cysteines are rendered incapable of disulfide linkage, all said cysteines are normally capable of intermolecular (preferably inter-heavy chain) disulfide linkage. In some embodiments of antibodies wherein two or more cysteines are rendered incapable of disulfide linkage, at least one of said cysteines is normally capable of intermolecular (for example, inter-heavy chain) disulfide linkage. In some embodiments, said intermolecular disulfide linkage is between cysteines of two immunoglobulin heavy chains.

In antibodies and methods of the invention, a cysteine residue can be rendered incapable of forming a disulfide linkage by any of a number of methods and techniques known in the art. For example, a hinge region cysteine that is normally capable of forming a disulfide linkage may be deleted. In another example, a cysteine residue of the hinge region that is normally capable of forming a disulfide linkage may be substituted with another amino acid, such as, for example, serine. In some embodiments, a hinge region cysteine residue may be modified such that it is incapable of disulfide bonding.

Antibodies of the invention can be of any of a variety of forms. For example, in one embodiment, an antibody of the invention is a full-length antibody, which preferably comprises a heavy chain and a light chain. In one aspect, the invention provides an antibody that is humanized. In another aspect, the invention provides a human antibody. In another aspect, the invention provides a chimeric antibody. An antibody of the invention may also be an antibody fragment, such as, for example, an Fc or Fc fusion polypeptide. An Fc fusion polypeptide generally comprises an Fc sequence (or fragment thereof) fused to a heterologous polypeptide sequence (such as an antigen binding domain, such as a receptor extracellular domain (ECD) fused to an immunoglobulin Fc sequence. For example, in one embodiment, an Fc fusion polypeptide comprises a VEGF binding domain, which may be a VEGF receptor, which includes flt, flk, etc. In another example, an Fc fusion polypeptide comprises a CD20 binding domain. In one example, an Fc fusion polypeptide comprises a tissue factor binding domain. In one example, an Fc fusion polypeptide comprises a HER2 or EGF receptor binding domain. In one example, an Fc fusion polypeptide comprises a hepatocyte growth factor receptor binding domain. In some embodiments, an antibody of the invention comprises a heavy chain constant domain sequence and a light chain constant domain sequence. In some embodiments, an antibody of the invention does not contain an added, substituted or modified amino acid in the Fc region (for example the hinge region) that is capable of intermolecular disulfide linkage. Generally, the Fc portion (or hinge region) of an antibody of the invention is not capable of an inter-heavy chain disulfide linkage. In one embodiment, an antibody of the invention does not comprise a modification (for example, but not limited to, insertion of one or more amino acids to, for e.g., form a dimerization sequence such as leucine zipper) that enables or enhances inter-heavy chain dimerization or multimerization.

An antibody of the invention can be of any isotype that comprises a hinge region, for e.g., IgG (including IgG1, IgG2, IgG3, IgG4). In some embodiments, the hinge region of an antibody of the invention is of an immunoglobulin selected from the group consisting of IgG1, IgG2, IgG3, IgG4.

Antibodies of the invention find a variety of uses in a variety of settings. For example, in one aspect, an antibody of the invention is a therapeutic antibody. An antibody of the invention can exert its therapeutic effect by any of a variety mechanisms. For example, an antibody of the invention may be an agonist antibody. In another example, an antibody of the invention may be an antagonistic antibody. In yet another example, an antibody of the invention may be a blocking antibody. In another example, an antibody of the invention is a neutralizing antibody.

Generally, and preferably, an antibody of the invention and its wild type counterpart antibody are substantially similar in certain biological/physiological characteristics but not in others, in particular with respect to effector functions. Generally, an antibody of the invention and its wild type counterpart comprise substantially similar antigen binding and/or disease fighting capabilities. In some embodiments, an antibody of the invention and its wild type counterpart have substantially similar FcRn binding capabilities. In some embodiments, an antibody of the invention and its wild type counterpart have substantially similar pharmacokinetic and/or pharmacodynamic characteristics/values.

Any of a number of host cells can be used in methods of the invention. Such cells are known in the art (some of which are described herein) or can be determined empirically using routine techniques known in the art. For e.g., a host cell is generally eukaryotic, for e.g. a mammalian cell such as the Chinese hamster ovary (CHO) cell.

Antibodies of the invention generally retain the antigen binding capability of their wild type counterparts. Thus, antibodies of the invention are capable of binding, preferably specifically, to antigens. Such antigens include, for example, tumor antigens, cell survival regulatory factors, cell proliferation regulatory factors, molecules associated with (for e.g., known or suspected to contribute functionally to) tissue development or differentiation, cell surface molecules, lymphokines, cytokines, molecules involved in cell cycle regulation, molecules involved in vasculogenesis and molecules associated with (for e.g., known or suspected to contribute functionally to) angiogenesis. An antigen to which an antibody of the invention is capable of binding may be a member of a subset of one of the above-mentioned categories, wherein the other subset(s) of said category comprise other molecules/antigens that have a distinct characteristic (with respect to the antigen of interest). An antigen of interest may also be deemed to belong to two or more categories. For example, in one embodiment, the invention provides an antibody that binds, preferably specifically, a tumor antigen that is not a cell surface molecule. In one embodiment, a tumor antigen is a cell surface molecule, such as a receptor polypeptide. In another example, in some embodiments, an antibody of the invention binds, preferably specifically, a tumor antigen that is not a cluster differentiation factor. In another example, an antibody of the invention is capable of binding, preferably specifically, to a cluster differentiation factor, which in some embodiments is not, for example, CD3 or CD4. In some embodiments, an antibody of the invention is an anti-VEGF antibody. In another example, an antibody of the invention is an anti-Tissue Factor antibody. In another example, an antibody of the invention is anti-CD20 antibody. In another example, an antibody of the invention is an anti-HER2 antibody. In another example, an antibody of the invention is an anti-EGFR antibody. In another example, an antibody of the invention is an anti-hepatocyte growth factor receptor antibody.

Antibodies of the invention are generally glycosylated. For e.g., an antibody of the invention may be glycosylated as a normal consequence of expression in a eukaryotic host cell, for e.g. a mammalian cell such as CHO. In one embodiment, glycosylation pattern of an antibody of the invention is substantially similar or identical to the glycosylation pattern of its wild type counterpart as determined by MALDI-TOF-MS analysis (which may be preceded by release of oligosaccharides, for e.g. by using a suitable enzyme such as N-glycosidase F). In one embodiment, an antibody of the invention comprises two N-linked oligosaccharides in the Fc region.

An antibody of the invention may be conjugated with a heterologous moiety. Any heterologous moiety would be suitable so long as its conjugation to the antibody does not substantially reduce a desired function and/or characteristic of the antibody. For example, in some embodiments, an immunoconjugate comprises a heterologous moiety which is a cytotoxic agent. In some embodiments, said cytotoxic agent is selected from the group consisting of a radioactive isotope, a chemotherapeutic agent and a toxin. In some embodiments, said toxin is selected from the group consisting of calichemicin, maytansine and trichothene. In some embodiments, an immunoconjugate comprises a heterologous moiety which is a detectable marker. In some embodiments, said detectable marker is selected from the group consisting of a radioactive isotope, a member of a ligand-receptor pair, a member of an enzyme-substrate pair and a member of a fluorescence resonance energy transfer pair.

In one aspect, the invention provides compositions comprising an antibody of the invention and an acceptable carrier (e.g., a pharmaceutically acceptable carrier). In one embodiment, the antibody is conjugated to a heterologous moiety.

In another aspect, the invention provides articles of manufacture comprising a container and a composition contained therein, wherein the composition comprises an antibody of the invention. In some embodiments, these articles of manufacture further comprise instruction for using said composition. In one embodiment, the antibody is provided in a therapeutically effective amount.

In yet another aspect, the invention provides polynucleotides encoding an antibody of the invention.

In one aspect, the invention provides recombinant vectors for expressing an antibody of the invention.

In one aspect, the invention provides host cells comprising a polynucleotide or recombinant vector of the invention. Preferably, a host cell is a eukaryotic cell, for example a mammalian cells such as CHO.

In one aspect, the invention provides methods of treating or delaying progression of a disease comprising administering to a subject having the disease an antibody of the invention effective in treating or delaying progression of the disease, wherein the antibody is modified such that inter-heavy chain disulfide linkages are substantially reduced or eliminated. Generally and preferably, the antibody is produced in a eukaryotic, such as mammalian, host cell. In one embodiment, the disease is a tumor or cancer. In one embodiment, the disease is an immunological discorder, for e.g. an autoimmune disease, for e.g., rheumatoid arthritis, immune thrombocytopenic purpura, systemic lupus erythematosus, etc. In another embodiment, the disease is associated with abnormal vascularization (such as angiogenesis).

In one aspect, the invention provides use of an antibody of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a nucleic acid of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of an expression vector of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a host cell of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of an article of manufacture of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides use of a kit of the invention in the preparation of a medicament for the therapeutic and/or prophylactic treatment of a disease, such as a cancer, a tumor, a cell proliferative disorder, an immune (such as autoimmune) disorder and/or an angiogenesis-related disorder.

In one aspect, the invention provides a method of inhibiting cell proliferation, said method comprising contacting a cell or tissue with an effective amount of an antibody of the invention, whereby cell proliferation is inhibited.

In one aspect, the invention provides a method of treating a pathological condition, said method comprising administering to the subject an effective amount of an antibody of the invention, whereby said condition is treated.

In one aspect, the invention provides a method of inhibiting the growth of a cell, said method comprising contacting said cell with an antibody of the invention thereby causing an inhibition of growth of said cell.

In one aspect, the invention provides a method of therapeutically treating a mammal having a cancerous tumor, said method comprising administering to said mammal an effective amount of an antibody of the invention, thereby effectively treating said mammal.

In one aspect, the invention provides a method for treating or preventing a cell proliferative disorder, said method comprising administering to a subject an effective amount of an antibody of the invention, thereby effectively treating or preventing said cell proliferative disorder. In one embodiment, said proliferative disorder is cancer.

In one aspect, the invention provides a method for inhibiting the growth of a cell, wherein growth of said cell is at least in part dependent upon a growth potentiating effect of a target molecule, said method comprising contacting said cell with an effective amount of an antibody of the invention that inhibits a biological function of said target molecule (e.g., by binding to said molecule), thereby inhibiting the growth of said cell.

A method of therapeutically treating a tumor in a mammal, wherein the growth of said tumor is at least in part dependent upon a growth potentiating effect of a target molecule, said method comprising contacting said cell with an effective amount of an antibody of the invention that inhibits a biological function of said target molecule (e.g., by binding to said molecule), thereby effectively treating said tumor.

Methods of the invention can be used to affect/modulate any suitable pathological state, for example, cells and/or tissues associated with dysregulation of a cellular signaling pathway. In one embodiment, a cell that is targeted in a method of the invention is a cancer cell. For example, a cancer cell can be one selected from the group consisting of a breast cancer cell, a colorectal cancer cell, a lung cancer cell, a papillary carcinoma cell (for e.g., of the thyroid gland), a colon cancer cell, a pancreatic cancer cell, an ovarian cancer cell, a cervical cancer cell, a central nervous system cancer cell, an osteogenic sarcoma cell, a renal carcinoma cell, a hepatocellular carcinoma cell, a bladder cancer cell, a gastric carcinoma cell, a head and neck squamous carcinoma cell, a prostate cancer cell, a lymphoma cell, a melanoma cell and a leukemia cell. In one embodiment, a cell that is targeted in a method of the invention is a hyperproliferative and/or hyperplastic cell. In one embodiment, a cell that is targeted in a method of the invention is a dysplastic cell. In yet another embodiment, a cell that is targeted in a method of the invention is a metastatic cell.

Methods of the invention can further comprise additional treatment steps. For example, in one embodiment, a method further comprises a step wherein a targeted cell and/or tissue (for e.g., a cancer cell) is exposed to radiation treatment or a chemotherapeutic agent.

In one embodiment of methods of the invention, a cell that is targeted (e.g., a cancer cell) is one in which amount and/or activity of a molecule inhibited (e.g., bound) by an antibody of the invention is enhanced as compared to a normal cell of the same tissue origin. In one embodiment, a method of the invention causes the death of a targeted cell. For example, contact with an antagonist antibody of the invention may result in a cell's inability to effect cellular signal transduction, thereby causing, for example, cell death.

In one embodiment of methods of the invention, therapeutic efficacy does not depend on effector function activity of a therapeutic antibody. In one embodiment, therapeutic efficacy is enhanced by using a therapeutic antibody that substantially lacks effector function activity. In one embodiment, a method of the invention relates to treating a pathological condition for which presence of effector function activity associated with a therapeutic antibody would be deemed to be clinically/therapeutically deleterious or undesirable.

### BRIEF DESCRIPTION OF THE DRAWINGS

FIG. 1: SDS-PAGE gel of anti-HER-2 and anti-TFIgG₁ with and without mutation in the hinge region. Lanes 2, 3 and 5 show antibody monomer without disulfide bonds.

FIG. 2: Native PAGE analysis of anti-TF IgG1 with and without the cysteine to serine mutation.

FIG. 3: Glycan composition assessed by MALDI/TOF-MS for different cell lines with and without the mutation in the hinge region showing no detectable impact on glycosylation profiles. Small differences that can be seen between cell lines is anticipated and within limits of expected clone-to-clone variations.

FIG. 4: FcRn binding of anti-TF IgG1 with and without cysteine residues in the hinge region showing no difference in their ability to bind to FcRn.

FIG. 5: Prothrombin time of normal human plasma with anti-TF IgG1 expressed with and without the cysteine to serine mutation in the hinge region showing no statistically significant difference in the time to clot formation expressed as fold prolongation (two-fold prolongation of the clotting time was measured at 25 μg/ml anti-TF IgG1 and 30 μg/ml anti-TF IgG1 hinge variant).

FIG. 6: Clearance of anti-TF IgG1 with wild type hinge (8.24±0.55 ml/day/kg) is similar to anti-TF IgG1 comprising variant hinge (10.47±2.62 ml/day/kg) for CHO-produced antibodies. Legend: E. coli hingeless (hinge variant); E. coli hinged (wild type hinge); CHO hingeless (hinge variant); CHO hinged (wild type hinge).

FIG. 7: C1q binding of anti-TF IgG1 with and without the mutation in the hinge region expressed in CHO as well as in E. Coli. Anti-TF IgG1 with the variant hinge region showed reduced binding capability compared to wild type antibodies. Rituxan (a commercially available anti-CD20 antibody) was run as a positive control case.

FIG. 8: C1q binding of anti-HER-2 with and without the two disulfide bonds in the hinge region expressed in CHO and E. coli. Anti-HER-2 without the mutation produced by a newly transfected cell line showed similar binding compared to the commercially available Herceptin. However, binding of anti-HER-2 with the variant hinge region is reduced to a level that is similar to the binding capacity of anti-HER-2 expressed in E. Coli.

FIG. 9: FcγRIa binding of anti-TF IgG1 with and without the mutation in the hinge region expressed in CHO as well as in E. coli. Rituxan and anti-TF IgG1 showed similar binding capacities. However, anti-TF IgG1 with the variant hinge region bound less than its wild type counterpart. As expected, full length anti-TF IgG1 expressed in E. coli with and without the mutation showed a significant decrease in binding to the FcγIa receptor.

FIG. 10: FcγRIa binding of anti-HER-2 with and without cysteine residues. Anti-HER-2 and Rituxan that were used as control cases showed similar binding capabilities. Conversely, anti-HER-2 with variant hinge region (cysteine to serine mutations) showed reduced binding. As expected, a significant reduction in binding capability could be observed for E. coli-expressed anti-HER-2.

FIG. 11: FcγRIIa binding of anti-TFIgG₁ with and without cysteine residues in the hinge region. Rituxan and a myeloma IgG₁ were used as control cases. Anti-TFIgG₁ without disulfide bonds in the hinge region showed a significant drop in its binding capacity. However, material expressed in E. coli with and without cysteine residues showed an even further decrease in binding to the FcγIIa receptor.

FIG. 12: FcγRIIa binding of anti-HER-2 with and without cysteine residues. Herceptin and Rituxan that were used as control cases showed similar binding capabilities. However, anti-HER-2 with the mutation in the hinge region as well as full length anti-HER-2 expressed in E. coli showed a dramatic decrease in binding to the receptor.

FIG. 13: FcγRIIb binding of anti-HER-2 with and without cysteine residues. Anti-HER-2 with the mutation in the hinge region expressed either in CHO or E. coli showed a significant decrease in binding compared to Rituxan and Herceptin.

FIG. 14: FcγRIIIa binding (high affinity allotype V158 and low affinity allotype F158) of anti-TF IgG1. Rituxan and a myeloma IgG₁ were used as a positive control. Binding of anti-TF IgG1 with mutation in the hinge region (i.e., without interchain disulfide bonds) to FcγRIIIa was dramatically reduced compared to material without the mutation. Both full length anti-TF IgG1 molecules expressed in E. coli showed only minimal amount of binding.

FIG. 15: FcγRIIIa (F158) binding of anti-HER-2 with and without the cysteine residues. Binding capability of anti-HER-2 with the mutation in the hinge region is significantly reduced and showed similar binding characteristics compared to anti-HER-2 produced in E. coli.

FIG. 16: FcγRIIIa (V158) binding of anti-HER-2 with and without the hinge variation. Binding profile of V158 allotype appeared to be similar to binding of allotype F158. Reduced binding capabilities of anti-HER-2 without the cysteine residues was observed.

FIGS. 17A & B: PBMC cell ADCC of SKBR3 cells. PBMC cells were isolated from buffy coat material ordered from Stanford Blood Bank. CHO as well as E. coli-derived hinge variant anti-HER-2 showed a significant decrease in cytotoxicity at various antibody concentrations compared to Herceptin reference material. FIG. 17A depicts data in graphical form. FIG. 17B depicts data in the form of numerical values.

FIG. 18: PBMC cell ADCC of SKBR3 cells. PBMC cells were isolated from fresh blood. Cytotoxicity of variant hinge anti-HER-2 expressed in CHO cells was significantly reduced. E. coli-derived anti-HER-2 with the mutation in the hinge region showed no detectable activity compared to reference Herceptin material. FIG. 18A depicts data in graphical form. FIG. 18B depicts data in the form of numerical values.

FIG. 19: Mean tumor volume of mammary tumor transplants in beige nude mice after 4 weeks exposure to anti-HER-2 antibody (30 mg/kg: single dose, 10 mg/kg: administered once per week for 4 weeks). Complete responses (annotated in the figure as “CR”) could be observed for hinge variant anti-HER2 treated at the 30 mg/kg dose, similar to Herceptin (a commercially available anti-HER2 antibody).

### MODES FOR CARRYING OUT THE INVENTION

The invention provides methods, compositions, kits and articles of manufacture for using immunoglobulins, preferably antibodies, comprising an alteration that reduces or eliminates the ability of heavy chains to form intermolecular (inter-heavy chain) disulfide linkages. Preferably these immunoglobulins comprise an alteration of at least one disulfide-forming cysteine residue such that the cysteine residue is incapable of forming a disulfide linkage. In one aspect, said cysteine(s) is of the hinge region of the heavy chain (thus, such hinge regions are referred to herein as “variant hinge region”). Generally and preferably, the hinge region of the immunoglobulin is mutated such that inter-heavy chain disulfide linkages are substantially reduced or eliminated. In some aspects, such immunoglobulins lack the complete repertoire of heavy chain cysteine residues that are normally capable of forming intermolecular (inter-heavy chain) disulfide linkages. Generally and preferably, the disulfide linkage formed by the cysteine residue(s) that is altered (i.e., rendered incapable of forming disulfide linkages) is one that, when not present in an antibody, does not result in a substantial loss of the therapeutic utility of the immunoglobulin (for e.g., tumor antigen targeting/specificity, efficacy, in vivo stability, etc.). Generally, the cysteine residue(s) that is rendered incapable of forming disulfide linkages is a cysteine of the hinge region of a heavy chain. Contrary to art teachings, it is herein shown that immunoglobulins comprising variant hinge regions in which at least one cysteine is incapable of disulfide linkage formation nonetheless possess essentially the same, and in certain contexts improved, physicochemical and/or therapeutic capabilities as compared to wild type immunoglobulins. Antibodies used in methods of the invention comprise an incomplete repertoire or a complete absence of the disulfide linkages normally formed by cysteines, in particular those formed by hinge cysteines. Details of methods, compositions, kits and articles of manufacture of the invention are provided herein.

General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988).

Definitions

The term “vector,” as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR.sub.2 (“amidate”), P(O)R, P(O)OR′, CO or CH.sub.2 (“formacetal”), in which each R or R′ is independently H or substituted or unsubstituted alkyl (1-20 C.) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

“Secretion signal sequence” or “signal sequence” refers to a nucleic acid sequence encoding a short signal peptide that can be used to direct a newly synthesized protein of interest through a cellular membrane, usually the inner membrane or both inner and outer membranes of prokaryotes. As such, the protein of interest such as the immunoglobulin light or heavy chain polypeptide is secreted into the periplasm of the prokaryotic host cells or into the culture medium. The signal peptide encoded by the secretion signal sequence may be endogenous to the host cells, or they may be exogenous, including signal peptides native to the polypeptide to be expressed. Secretion signal sequences are typically present at the amino terminus of a polypeptide to be expressed, and are typically removed enzymatically between biosynthesis and secretion of the polypeptide from the cytoplasm. Thus, the signal peptide is usually not present in a mature protein product.

The term “host cell” (or “recombinant host cell”), as used herein, is intended to refer to a cell that has been genetically altered, or is capable of being genetically altered by introduction of an exogenous polynucleotide, such as a recombinant plasmid or vector. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

The terms “antibody” and “immunoglobulin” are used interchangeably in the broadest sense and include monoclonal antibodies (for e.g., full length or intact monoclonal antibodies), polyclonal antibodies, multivalent antibodies, and multispecific antibodies (e.g., bispecific antibodies so long as they exhibit the desired biological activity). Antibodies and immunoglobulins of the invention comprise mutations in the hinge region that negatively affect formation of disulfide linkages. In one aspect, antibodies and immunoglobulins of the invention comprise a hinge region in which at least one cysteine residue is rendered incapable of forming a disulfide linkage, wherein the disulfide linkage is preferably intermolecular, preferably between two heavy chains. A hinge cysteine can be rendered incapable of forming a disulfide linkage by any of a variety of suitable methods known in the art, some of which are described herein, including but not limited to deletion of the cysteine residue or substitution of the cysteine with another amino acid.

Depending on the amino acid sequences of the constant domains of their heavy chains, antibodies (immunoglobulins) can be assigned to different classes. There are five major classes of immunoglobulins: IgA, IgD, IgE, IgG and IgM, and several of these may be further divided into subclasses (isotypes), e.g., IgG-1, IgG-2, IgA-1, IgA-2, and etc. The heavy chain constant domains that correspond to the different classes of immunoglobulins are called α, δ, ε, γ, and μ, respectively. The subunit structures and three-dimensional configurations of different classes of immunoglobulins are well known and described generally in, for example, Abbas et al. Cellular and Mol. Immunology, 4th ed. (2000). An antibody may be part of a larger fusion molecule, formed by covalent or non-covalent association of the antibody with one or more other proteins or peptides.

The terms “full length antibody,” “intact antibody” and “whole antibody” are used herein interchangeably, and are intended to refer to an antibody in its substantially intact form (for e.g., in contrast to antibody fragments such as Fab in which substantially or all of the Fc portion of the heavy chain is missing). The terms particularly refer to an antibody with heavy chains that comprise the Fc region. An antibody variant of the invention can be a full length antibody. A full length antibody can be, for e.g., human, humanized and/or affinity matured.

A “biologically active” or “functional” immunoglobulin is one capable of exerting one or more of its natural activities in structural, regulatory, biochemical or biophysical events. For example, a biologically active antibody may have the ability to specifically bind an antigen and the binding may in turn elicit or alter a cellular or molecular event such as signaling transduction or enzymatic activity. A biologically active antibody may also block ligand activation of a receptor or act as an agonist antibody. The capability of an antibody to exert one or more of its natural activities depends on several factors, including proper folding and assembly of the polypeptide chains. Preferably, a “biologically active” antibody is an antibody that is intended to be used primarily to achieve a biological/physiological response that would lead to therapeutic effects, in vivo or ex vivo, for example to alleviate or treat diseases. Thus, for example, a “biologically active” antibody preferably does not include an antibody produced solely as a reference or control antibody used as a comparitor. It should also be noted that an antibody of the invention which is “biologically active” or “functional” does not necessarily retain all of the functions/capabilities of its wild type form (e.g., as described extensively herein, certain effector functions may be reduced or eliminated).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies that contain minimal sequence derived from non-human immunoglobulin. Generally, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally will also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues.

An “affinity matured” antibody is one with one or more alterations in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term “Fc region” is used to define the C-terminal region of an immunoglobulin heavy chain which may be generated by papain digestion of an intact antibody. The Fc region may be a native sequence Fc region or a variant Fc region. Although the boundaries of the Fc region of an immunoglobulin heavy chain might vary, the human IgG heavy chain Fc region is usually defined to stretch from an amino acid residue at about position Cys226, or from about position Pro230, to the carboxyl-terminus of the Fc region. The Fc region of an immunoglobulin generally comprises two constant domains, a CH2 domain and a CH3 domain, and optionally comprises a CH4 domain. By “Fc region chain” herein is meant one of the two polypeptide chains of an Fc region.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell.

The terms “Fc receptor” and “FcR” are used to describe a receptor that binds to the Fc region of an antibody. For e.g., an FcR can be a native sequence human FcR. Generally, an FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Immunoglobulins of other isotypes can also be bound by certain FcRs (see, for e.g., Janeway et al., Immuno Biology: the immune system in health and disease, (Elsevier Science Ltd., NY) (4th ed., 1999)). Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (reviewed in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. In some contexts, the term also includes the neonatal receptor, FcRn, which is responsible for the transfer of maternal IgGs to the fetus (Guyer et al., J. Immunol. 117:587 (1976); and Kim et al., J. Immunol. 24:249 (1994)).

The “hinge region,” and variations thereof, as used herein, includes the meaning known in the art, which is illustrated in, for example, Janeway et al., Immuno Biology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

An “altered” or “variant” heavy chain, as used herein, generally refers to a heavy chain with reduced disulfide linkage capability, for e.g., wherein at least one cysteine residue has been rendered incapable of disulfide linkage formation. As described herein, in general an antibody of the invention substantially lacks inter-heavy chain disulfide linkages. Generally, at least one, and in some examples up to all, of the cysteines in the hinge region that normally form inter-heavy chain disulfide linkages are altered.

As used herein, the phrase “wild type counterpart(s)” or variations thereof, refers to antibodies that differ from the antibodies of the invention in the hinge region primarily or solely with respect to the extent they are capable of disulfide linkage formation, for e.g., as determined by whether one or more hinge cysteines is rendered incapable of forming disulfide linkages.

The phrase “amount of an activity (e.g., ADCC, receptor binding, CDC, complement binding, etc.) of a variant immunoglobulin or antibody is less than (or substantially reduced compared to) the amount of the same activity of its wild type counterpart”, and variations thereof, as used herein, means the difference in amount of detectable activity of a variant immunoglobulin or antibody of the invention and the amount of the activity exhibited by the wild type form is statistically significant as evident to one skilled in the art. As would be understood in the art, amount of an activity may be determined quantitatively or qualitatively, so long as a comparison between an immunoglubin or antibody of the invention and its wild type counterpart can be done. The activity can be measured or detected according to any assay or technique known in the art, including, for e.g., those described herein. The amount of activity for an immunoglobulin or antibody of the invention and its wild type counterpart can be determined in parallel or in separate runs.

The phrase “substantially similar”, “substantially identical”, “substantially the same”, and variations thereof, as used herein, denotes a sufficiently high degree of similarity between two numeric values (generally one associated with an antibody of the invention and the other associated with its wild type counterpart) such that one of skill in the art would consider the difference between the two values to be of little or no biological significance within the context of the biological, physical or quantitation characteristic measured by said values. The difference between said two values is preferably less than about 50%, preferably less than about 40%, preferably less than about 30%, preferably less than about 20%, preferably less than about 10% as a function of the value for the wild type counterpart.

“Complement dependent cytotoxicity” and “CDC” refer to the lysing of a target in the presence of complement. The complement activation pathway is initiated by the binding of the first component of the complement system (C1q) to a molecule (e.g. an antibody) complexed with a cognate antigen.

“Binding affinity” generally refers to the strength of the sum total of noncovalent interactions between a single binding site of a molecule (e.g., an antibody) and its binding partner (e.g., an antigen or FcRn receptor). The affinity of a molecule X for its partner Y can generally be represented by the dissociation constant (Kd). Affinity can be measured by common methods known in the art, including those described herein. Low-affinity antibodies bind antigen (or FcRn receptor) weakly and tend to dissociate readily, whereas high-affinity antibodies bind antigen (or FcRn receptor) more tightly and remain bound longer.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY117018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®). In addition, such definition of chemotherapeutic agents includes bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small-molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

A “blocking” antibody or an “antagonist” antibody is one which inhibits or reduces biological activity of the antigen it binds. Such blocking can occur by any means, e.g. by interfering with: ligand binding to the receptor, receptor complex formation, tyrosine kinase activity of a tyrosine kinase receptor in a receptor complex and/or phosphorylation of tyrosine kinase residue(s) in or by the receptor. For example, a VEGF antagonist antibody binds VEGF and inhibits the ability of VEGF to induce vascular endothelial cell proliferation. Preferred blocking antibodies or antagonist antibodies substantially or completely inhibit the biological activity of the antigen.

An “agonist antibody” is an antibody which binds and activates antigen such as a receptor. Generally, the receptor activation capability of the agonist antibody will be at least qualitatively similar (and may be essentially quantitatively similar) to a native agonist ligand of the receptor.

An antibody of the invention “which binds antigen essentially as effectively as” its wild type counterpart antibody is one capable of binding that antigen with affinity and/or avidity that is within about 10 fold, preferably about 5 fold, and more preferably about 2 fold, of the binding affinity and/or avidity of the wild type counterpart antibody, for example when binding affinity is expressed as Kd, Ka, and/or EC₅₀ values.

A “tumor antigen,” as used herein, includes the meaning known in the art, which includes any molecule expressed on (or associated with the development of) a tumor cell that is known or thought to contribute to a tumorigenic characteristic of the tumor cell. Numerous tumor antigens are known in the art. Whether a molecule is a tumor antigen can also be determined according to techniques and assays well known to those skilled in the art, such as for example clonogenic assays, transformation assays, in vitro or in vivo tumor formation assays, gel migration assays, gene knockout analysis, etc.

A “disorder” or “disease” is any condition that would benefit from administration of an immunoglobulin or antibody of the invention to a subject or treatment of the subject with said immunoglobulin or antibody, wherein the subject is known or suspected of having the disorder or disease. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, angiogenic and immunologic disorders.

An “autoimmune disease” herein is a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitis); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

The terms “cancer” and “cancerous” refer to or describe the physiological condition in mammals that is typically characterized by unregulated cell growth/proliferation. Examples of cancer include but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

Dysregulation of angiogenesis can lead to many disorders that can be treated by compositions and methods of the invention. These disorders include both non-neoplastic and neoplastic conditions. Neoplastics include but are not limited those described above. Non-neoplastic disorders include but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or disorder.

An “effective amount” refers to an amount effective, at dosages and for periods of time necessary, to achieve the desired therapeutic or prophylactic result. A “therapeutically effective amount” of the antibody may vary according to factors such as the disease state, age, sex, and weight of the individual, and the ability of the antibody to elicit a desired response in the individual. A therapeutically effective amount is also one in which any toxic or detrimental effects of the antibody are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

Methods of the Invention

In numerous pathological conditions, a therapeutic antibody may effect its therapeutic action without involving immune system-mediated activities, such as the effector functions ADCC, phagocytosis and CDC. In such situations, it is desirable to engineer the antibody such that such activities are substantially reduced or eliminated. Unfortunately, there are numerous challenges towards achieving such a goal. For e.g., alteration or elimination of all or part of the Fc region, which is involved in numerous effector functions, may also result in unwanted alteration of effector functions that are desirable (such as binding to FcRn and clearance in vivo). That is, it would be advantageous to engineer an antibody that exhibits a subset, but not all, of wild type effector functions, while retaining its therapeutic utility. Moreover, it would be even more advantageous if the engineered antibody can be produced without a substantial reduction in yield compared to its wild type counterpart. The present invention provides these antibodies, which are demonstrated to possess substantially similar therapeutic efficacy as their wild type counterparts.

Accordingly, in one aspect, the invention provides methods of treating a disease using a biologically active immunoglobulin, said methods comprising administering to a subject in need of treatment an antibody in which at least one, at least two, at least three, at least four, or between two and eleven inter-heavy chain disulfide linkages are eliminated, whereby the disease is treated. For e.g., the antibody comprises a variant hinge region of an immunoglobulin heavy chain, wherein at least one cysteine of said variant hinge region is rendered incapable of forming a disulfide linkage.

It is further anticipated that any cysteine in an immunoglobulin heavy chain can be rendered incapable of disulfide linkage formation, similarly to the hinge cysteines described herein, provided that such alteration does not substantially reduce the therapeutic utility of the immunoglobulin. For example, IgM and IgE lack a hinge region, but each contains an extra heavy chain domain; at least one (in some embodiments, all) of the cysteines of the heavy chain can be rendered incapable of disulfide linkage formation in antibodies used in methods of the invention so long as it does not substantially reduce the therapeutic function of the antibody which comprises the heavy chain.

Heavy chain hinge cysteines are well known in the art, as described in, for example, “Sequences of proteins of immunological interest” by Kabat. As is known in the art, the number of hinge cysteines varies depending on the class and subclass of immunoglobulin. See, for example, Janeway, “Immunobiology”, 4th Ed., (Garland Publishing, NY). For example, in human IgG1s, there are two hinge cysteines that are separated by two prolines, and these are normally paired with their counterparts on an adjacent heavy chain in intermolecular disulfide linkages. Other examples include human IgG2 which contains 4 hinge cysteines, IgG3 which contains 11 hinge cysteines, and IgG4 which contains 2 hinge cysteines. Accordingly, in one embodiment, an antibody used in methods of the invention comprises a variant hinge region, wherein at least one cysteine of said variant hinge region is rendered incapable of forming a disulfide linkage. In another embodiment, methods of the invention comprise using an antibody comprising a variant hinge region, wherein at least two cysteines of said variant hinge region are rendered incapable of forming a disulfide linkage. In one embodiment, an antibody used in methods of the invention comprises a variant hinge region, wherein at least three cysteines of said variant hinge region are rendered incapable of forming a disulfide linkage. In one embodiment, an antibody used in methods of the invention comprises a variant hinge region, wherein from about two to about eleven cysteines of said variant hinge region are rendered incapable of forming a disulfide linkage. In one embodiment, an antibody used in methods of the invention comprises a variant hinge region, wherein at least four cysteines of said variant hinge region are rendered incapable of forming a disulfide linkage. In one embodiment, an antibody used in methods of the invention comprises a variant hinge region, wherein all cysteines of said variant hinge region are rendered incapable of forming a disulfide linkage.

Light chains and heavy chains constituting antibodies of the invention as used in methods of the invention may be encoded by and thus generated from a single polynucleotide or by separate polynucleotides.

Cysteines normally involved in disulfide linkage formation can be rendered incapable of forming disulfide linkages by any of a variety of methods known in the art, that would be evident to one skilled in the art in view of the criteria described herein. For example, a hinge cysteine can be substituted with another amino acid, such as serine, which is not capable of disulfide bonding. Amino acid substitution can be achieved by standard molecular biology techniques, such as site directed mutagenesis of the nucleic acid sequence encoding the hinge region that is to be modified. Suitable techniques include those described in Sambrook et al., supra. Other techniques for generating immunoglobulin with a variant hinge region include synthesizing an oligonucleotide comprising a sequence that encodes a hinge region in which the codon that encodes the cysteine that is to be substituted is replaced with a codon that encodes the substitute amino acid. This oligonucleotide can then be ligated into a vector backbone comprising other appropriate antibody sequences, such as variable regions and Fc sequences, as appropriate. Details of examples of these techniques are further described in the Examples section below. In another example, a hinge cysteine can be deleted. Amino acid deletion can be achieved by standard molecular biology techniques, such as site directed mutagenesis of the nucleic acid sequence encoding the hinge region that is to be modified. Suitable techniques include those described in Sambrook et al., supra. Other techniques for generating immunoglobulin with a variant hinge region include synthesizing an oligonucleotide comprising a sequence that encodes a hinge region in which the codon that encodes the cysteine that is to be modified is deleted. This oligonucleotide can then be ligated into a vector backbone comprising other appropriate antibody sequences, such as variable regions and Fc sequences, as appropriate.

Antigen Specificity

The present invention is applicable to antibodies of any appropriate antigen binding specificity. Preferably, the antibodies used in methods of the invention are specific to antigens that are biologically important polypeptides. More preferably, the antibodies of the invention are useful for therapy or diagnosis of diseases or disorders in a mammal. Antibodies of the invention include, but are not limited to blocking antibodies, agonist antibodies, neutralizing antibodies or antibody conjugates. Non-limiting examples of therapeutic antibodies include anti-VEGF, anti-IgE, anti-CD11, anti-CD18, anti-CD40, anti-tissue factor (TF), anti-HER2, and anti-TrkC antibodies. Antibodies directed against non-polypeptide antigens (such as tumor-associated glycolipid antigens) are also contemplated.

Where the antigen is a polypeptide, it may be a transmembrane molecule (e.g. receptor) or a ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; hepatocyte growth factor (HGF); c-met; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD3, CD4, CD8, CD19, CD20 and CD40; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

Antigens for antibodies encompassed by one embodiment of the present invention include CD proteins such as CD3, CD4, CD8, CD19, CD20, CD34, and CD46; members of the ErbB receptor family such as the EGF receptor, HER2, HER3 or HER4 receptor; cell adhesion molecules such as LFA-1, Mac1, p150.95, VLA-4, ICAM-1, VCAM, α4/β7 integrin, and αv/β3 integrin including either α or β subunits thereof (e.g. anti-CD11a, anti-CD18 or anti-CD11b antibodies); growth factors such as VEGF; tissue factor (TF); TGF-β; alpha interferon (α-IFN); an interleukin, such as IL-8; IgE; blood group antigens Apo2, death receptor; flk2/flt3 receptor; obesity (OB) receptor; mpl receptor; CTLA-4; protein C etc. In some embodiments, targets herein are VEGF, TF, CD19, CD20, CD40, TGF-β, CD11a, CD18, Apo2 and C24.

In some embodiments, an antibody of the invention is capable of binding specifically to a tumor antigen. In some embodiments, an antibody of the invention is capable of binding specifically to a tumor antigen wherein the tumor antigen is not a cluster differentiation factor (i.e., a CD protein). In some embodiments, an antibody of the invention is capable of binding specifically to a CD protein. In some embodiments, an antibody of the invention is capable of binding specifically to a CD protein other than CD3 or CD4. In some embodiments, an antibody of the invention is capable of binding specifically to a CD protein other than CD19 or CD20. In some embodiments, an antibody of the invention is capable of binding specifically to a CD protein other than CD40. In some embodiments, an antibody of the invention is capable of binding specifically to CD19 or CD20. In some embodiments, an antibody of the invention is capable of binding specifically to CD40. In some embodiments, an antibody of the invention is capable of binding specifically to CD11.

In one embodiment, an antibody of the invention is capable of binding specifically to a cell survival regulatory factor. In some embodiments, an antibody of the invention is capable of binding specifically to a cell proliferation regulatory factor. In some embodiments, an antibody of the invention is capable of binding specifically to a molecule involved in cell cycle regulation. In other embodiments, an antibody of the invention is capable of binding specifically to a molecule involved in tissue development or cell differentiation. In some embodiments, an antibody of the invention is capable of binding specifically to a cell surface molecule. In some embodiments, an antibody of the invention is capable of binding to a tumor antigen that is not a cell surface receptor polypeptide.

In one embodiment, an antibody of the invention is capable of binding specifically to a lymphokine. In another embodiment, an antibody of the invention is capable of binding specifically to a cytokine.

In one embodiment, antibodies of the invention are capable of binding specifically to a molecule involved in vasculogenesis. In another embodiment, antibodies of the invention are capable of binding specifically to a molecule involved in angiogenesis.

Soluble antigens or fragments thereof, optionally conjugated to other molecules, can be used as immunogens for generating antibodies. For transmembrane molecules, such as receptors, fragments of these molecules (e.g. the extracellular domain of a receptor) can be used as the immunogen. Alternatively, cells expressing the transmembrane molecule can be used as the immunogen. Such cells can be derived from a natural source (e.g. cancer cell lines) or may be cells which have been transformed by recombinant techniques to express the transmembrane molecule. Other antigens and forms thereof useful for preparing antibodies will be apparent to those in the art.

The antibodies of the present invention may be monospecific, bispecific, trispecific or of greater multispecificity. Multispecific antibodies may be specific to different epitopes of a single molecule or may be specific to epitopes on different molecules. Methods for designing and making multispecific antibodies are known in the art. See, e.g., Millstein et al. (1983) Nature 305:537-539; Kostelny et al. (1992) J. Immunol. 148:1547-1553; WO 93/17715.

Vectors, Host Cells and Recombinant Methods

For recombinant production of an antibody of the invention, the nucleic acid encoding it is isolated and inserted into a replicable vector for further cloning (amplification of the DNA) or for expression. DNA encoding the antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

(i) Signal Sequence Component

An antibody of the invention may be produced recombinantly not only directly, but also as a fusion polypeptide with a heterologous polypeptide, which is preferably a signal sequence or other polypeptide having a specific cleavage site at the N-terminus of the mature protein or polypeptide. The heterologous signal sequence selected preferably is one that is recognized and processed (i.e., cleaved by a signal peptidase) by the host cell. In mammalian cell expression, mammalian signal sequences as well as viral secretory leaders, for example, the herpes simplex gD signal, are available.

The DNA for such precursor region is ligated in reading frame to DNA encoding the antibody.

(ii) Origin of Replication

Generally, an origin of replication component is not needed for mammalian expression vectors. For example, the SV40 origin may typically be used only because it contains the early promoter.

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, where relevant, or (c) supply critical nutrients not available from complex media.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, etc.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity (e.g., ATCC CRL-9096).

Alternatively, host cells (particularly wild-type hosts that contain endogenous DHFR) transformed or co-transformed with DNA sequences encoding an antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

(iv) Promoter Component

Expression and cloning vectors usually contain a promoter that is recognized by the host organism and is operably linked to the antibody nucleic acid. Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., Nature 297:598-601 (1982) on expression of human β-interferon cDNA in mouse cells under the control of a thymidine kinase promoter from herpes simplex virus. Alternatively, the Rous Sarcoma Virus long terminal repeat can be used as the promoter.

(v) Enhancer Element Component

Transcription of a DNA encoding the antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, Nature 297:17-18 (1982) on enhancing elements for activation of eukaryotic promoters. The enhancer may be spliced into the vector at a position 5′ or 3′ to the antibody-encoding sequence, but is preferably located at a site 5′ from the promoter.

(vi) Transcription Termination Component

Expression vectors used in eukaryotic host cells will typically also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an antibody. One useful transcription termination component is the bovine growth hormone polyadenylation region. See WO94/11026 and the expression vector disclosed therein.

(vii) Selection and Transformation of Host Cells

Suitable host cells for cloning or expressing the DNA in the vectors herein include higher eukaryote cells described herein, including vertebrate host cells. Propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for antibody production and cultured in conventional nutrient media modified as appropriate for inducing promoters, selecting transformants, or amplifying the genes encoding the desired sequences.

(viii) Culturing the Host Cells

The host cells used to produce an antibody of this invention may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

(ix) Purification of Antibody

When using recombinant techniques, the antibody can be produced intracellularly, or directly secreted into the medium. If the antibody is produced intracellularly, as a first step, the particulate debris, either host cells or lysed fragments, are removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

Activity Assays

The immunoglobulins of the present invention can be characterized for their physical/chemical properties and biological functions by various assays known in the art. In one aspect of the invention, it is important to compare the variant hinge antibodies of the present invention to their wild type counterparts.

The purified immunoglobulins can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

In certain embodiments of the invention, the immunoglobulins produced herein are analyzed for their biological activity. In some embodiments, the immunoglobulins of the present invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays. An illustrative antigen binding assay is provided below in the Examples section.

In one embodiment, the present invention contemplates an altered antibody that possesses some but not all effector functions. The unique features of the antibody (i.e., having an intact or substantially intact Fc region, yet lacking some but not all effector functions) make it a desired candidate for many applications in which the half life of the antibody in vivo is important yet certain effector functions (such as complement and ADCC) are unnecessary or deleterious. In certain embodiments, the Fc activities of the produced immunoglobulin are measured to ensure that only the desirable properties are maintained. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). An example of an in vitro assay to assess ADCC activity of a molecule of interest is described in U.S. Pat. Nos. 5,500,362 or 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). C1q binding assays may also be carried out to confirm that the antibody is unable to bind C1q and hence lacks CDC activity. To assess complement activation, a CDC assay, for e.g. as described in Gazzano-Santoro et al., J. Immunol. Methods 202:163 (1996), may be performed. FcRn binding and in vivo clearance/half life determinations can also be performed using methods known in the art, for e.g. those described in the Examples section.

Humanized Antibodies

The present invention encompasses humanized antibodies. Various methods for humanizing non-human antibodies are known in the art. For example, a humanized antibody can have one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework for the humanized antibody (Sims et al. (1993) J. Immunol. 151:2296; Chothia et al. (1987) J. Mol. Biol. 196:901. Another method uses a particular framework derived from the consensus sequence of all human antibodies of a particular subgroup of light or heavy chains. The same framework may be used for several different humanized antibodies (Carter et al. (1992) Proc. Natl. Acad. Sci. USA, 89:4285; Presta et al. (1993) J. Immunol., 151:2623.

It is further important that antibodies be humanized with retention of high affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Antibody Variants

Amino acid sequence modification(s) of the antibodies described herein are contemplated. For example, it may be desirable to improve the binding affinity and/or other biological properties of the antibody. Amino acid sequence variants of the antibody are prepared by introducing appropriate nucleotide changes into the antibody nucleic acid, or by peptide synthesis. Such modifications include, for example, deletions from, and/or insertions into and/or substitutions of, residues within the amino acid sequences of the antibody. Any combination of deletion, insertion, and substitution is made to arrive at the final construct, provided that the final construct possesses the desired characteristics. The amino acid alterations may be introduced in the subject antibody amino acid sequence at the time that sequence is made.

A useful method for identification of certain residues or regions of the antibody that are preferred locations for mutagenesis is called “alanine scanning mutagenesis” as described by Cunningham and Wells (1989) Science, 244:1081-1085. Here, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed immunoglobulins are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative substitutions are shown in Table 2 under the heading of “preferred substitutions”. If such substitutions result in a change in biological activity, then more substantial changes, denominated “exemplary substitutions” in Table 2, or as further described below in reference to amino acid classes, may be introduced and the products screened.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | Leu |
| | Phe; Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | He; Leu; Met; Phe; | Leu |
| | Ala; Norleucine | |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

(1) non-polar: Ala (A), Val (V), Leu (L), Ile (I), Pro (P), Phe (F), Trp (W), Met (M)

(2) uncharged polar: Gly (G), Ser (S), Thr (T), Cys (C), Tyr (Y), Asn (N), Gln (Q)

(3) acidic: Asp (D), Glu (E)

(4) basic: Lys (K), Arg (R), His (H)

Alternatively, naturally occurring residues may be divided into groups based on common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

One type of substitutional variant involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g. 6-7 sites) are mutated to generate all possible amino acid substitutions at each site. The antibodies thus generated are displayed from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

It may be desirable to introduce one or more amino acid modifications in an Fc region of the immunoglobulin polypeptides of the invention, thereby generating a Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions including that of a hinge cysteine.

In accordance with this description and the teachings of the art, it is contemplated that in some embodiments, an antibody used in methods of the invention may comprise one or more alterations as compared to the wild type counterpart antibody, for e.g. in the Fc region, in addition to the hinge sequence mutation described herein. These antibodies would nonetheless retain substantially the same characteristics required for therapeutic utility as compared to their wild type counterpart. For e.g., it is thought that certain alterations can be made in the Fc region that would result in altered (i.e., either improved or diminished) C1q binding and/or Complement Dependent Cytotoxicity (CDC), for e.g., as described in WO99/51642. See also Duncan & Winter Nature 322:738-40 (1988); U.S. Pat. No. 5,648,260; U.S. Pat. No. 5,624,821; and WO94/29351 concerning other examples of Fc region variants.

Immunoconjugates

The invention also pertains to immunoconjugates, or antibody-drug conjugates (ADC), comprising an antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate).

The use of antibody-drug conjugates for the local delivery of cytotoxic or cytostatic agents, i.e. drugs to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) theoretically allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet pp. (Mar. 15, 1986):603-05; Thorpe, (1985) “Antibody Carriers Of Cytotoxic Agents In Cancer Therapy: A Review,” in Monoclonal Antibodies '84: Biological And Clinical Applications, A. Pinchera et al. (ed.s), pp. 475-506). Maximal efficacy with minimal toxicity is sought thereby. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al (2000) Jour. of the Nat. Cancer Inst. 92(19): 1573-1581; Mandler et al (2000) Bioorganic & Med. Chem. Letters 10:1025-1028; Mandler et al (2002) Bioconjugate Chem. 13:786-791), maytansinoids (EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). The toxins may effect their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

ZEVALIN® (ibritumomab tiuxetan, Biogen/Idec) is an antibody-radioisotope conjugate composed of a murine IgG1 kappa monoclonal antibody directed against the CD20 antigen found on the surface of normal and malignant B lymphocytes and ¹¹¹In or ⁹⁰Y radioisotope bound by a thiourea linker-chelator (Wiseman et al (2000) Eur. Jour. Nucl. Med. 27(7):766-77; Wiseman et al (2002) Blood 99(12):4336-42; Witzig et al (2002) J. Clin. Oncol. 20(10):2453-63; Witzig et al (2002) J. Clin. Oncol. 20(15):3262-69). Although ZEVALIN has activity against B-cell non-Hodgkin's Lymphoma (NHL), administration results in severe and prolonged cytopenias in most patients. MYLOTARG™ (gemtuzumab ozogamicin, Wyeth Pharmaceuticals), an antibody drug conjugate composed of a hu CD33 antibody linked to calicheamicin, was approved in 2000 for the treatment of acute myeloid leukemia by injection (Drugs of the Future (2000) 25(7):686; U.S. Pat. Nos. 4,970,198; 5,079,233; 5,585,089; 5,606,040; 5,693,762; 5,739,116; 5,767,285; 5,773,001). Cantuzumab mertansine (Immunogen, Inc.), an antibody drug conjugate composed of the huC242 antibody linked via the disulfide linker SPP to the maytansinoid drug moiety, DM1, is advancing into Phase II trials for the treatment of cancers that express CanAg, such as colon, pancreatic, gastric, and others. MLN-2704 (Millennium Pharm., BZL Biologics, Immunogen Inc.), an antibody drug conjugate composed of the anti-prostate specific membrane antigen (PSMA) monoclonal antibody linked to the maytansinoid drug moiety, DM1, is under development for the potential treatment of prostate tumors. The auristatin peptides, auristatin E (AE) and monomethylauristatin (MMAE), synthetic analogs of dolastatin, were conjugated to chimeric monoclonal antibodies cBR96 (specific to Lewis Y on carcinomas) and cAC10 (specific to CD30 on hematological malignancies) (Doronina et al (2003) Nature Biotechnology 21(7):778-784) and are under therapeutic development.

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothecene, and CC1065, and the derivatives of these toxins that have toxin activity, are also contemplated herein.

Maytansine and Maytansinoids

In one embodiment, an antibody (full length or fragments) of the invention is conjugated to one or more maytansinoid molecules.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

Maytansinoid-Antibody Conjugates

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansinoid drug, which could be increased by increasing the number of maytansinoid molecules per antibody molecule. The A7-maytansinoid conjugate showed low systemic cytotoxicity in mice.

Antibody-Maytansinoid Conjugates (Immunoconjugates)

Antibody-maytansinoid conjugates are prepared by chemically linking an antibody to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody-maytansinoid conjugates, including, for example, those disclosed in U.S. Pat. No. 5,208,020 or EP Patent 0 425 235 B1, and Chari et al., Cancer Research 52:127-131 (1992). The linking groups include disulfide groups, thioether groups, acid labile groups, photolabile groups, peptidase labile groups, or esterase labile groups, as disclosed in the above-identified patents, disulfide and thioether groups being preferred.

Conjugates of the antibody and maytansinoid may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio)propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hydroxymethyl, the C-15 position modified with a hydroxyl group, and the C-20 position having a hydroxyl group. In a preferred embodiment, the linkage is formed at the C-3 position of maytansinol or a maytansinol analogue.

Calicheamicin

Another immunoconjugate of interest comprises an antibody conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γ₁^{I}, α₂^{I}, α₃^{I}, N-acetyl-γ₁^{I}, PSAG and θ^{I}₁ (Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

Other Cytotoxic Agents

Other antitumor agents that can be conjugated to the antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for detection, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio- or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, .Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal, CRC Press 1989) describes other methods in detail.

Conjugates of the antibody and cytotoxic agent may be made using a variety of bifunctional protein coupling agents such as N-succinimidyl-3-(2-pyridyldithio)propionate (SPDP), succinimidyl-4-(N-maleimidomethyl)cyclohexane-1-carboxylate, iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl)hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The compounds of the invention expressly contemplate, but are not limited to, ADC prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A). See pages 467-498, 2003-2004 Applications Handbook and Catalog.

Preparation of Antibody Drug Conjugates

In the antibody drug conjugates (ADC) of the invention, an antibody (Ab) is conjugated to one or more drug moieties (D), e.g. about 1 to about 20 drug moieties per antibody, through a linker (L). The ADC of Formula I may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody.
Ab-(L-D)ₚ

Nucleophilic groups on antibodies include, but are not limited to: (i) N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol.

Antibody drug conjugates of the invention may also be produced by modification of the antibody to introduce electrophilic moieties, which can react with nucleophilic substituents on the linker reagent or drug. The sugars of glycosylated antibodies may be oxidized, e.g. with periodate oxidizing reagents, to form aldehyde or ketone groups which may react with the amine group of linker reagents or drug moieties. The resulting imine Schiff base groups may form a stable linkage, or may be reduced, e.g. by borohydride reagents to form stable amine linkages. In one embodiment, reaction of the carbohydrate portion of a glycosylated antibody with either glactose oxidase or sodium meta-periodate may yield carbonyl (aldehyde and ketone) groups in the protein that can react with appropriate groups on the drug (Hermanson, Bioconjugate Techniques). In another embodiment, proteins containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Antibody Derivatives

The antibodies of the present invention can be further modified to contain additional nonproteinaceous moieties that are known in the art and readily available. Preferably, the moieties suitable for derivatization of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymers are attached, they can be the same or different molecules. In general, the number and or type of polymers used for derivatization can be determined based on considerations including, but not limited to, the particular properties or functions of the antibody to be improved, whether the antibody derivative will be used in a therapy under defined conditions.

Pharmaceutical Formulations

Therapeutic formulations comprising an antibody of the invention are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of aqueous solutions, lyophilized or other dried formulations. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulation herein may also contain more than one active compound as necessary for the particular indication being treated, preferably those with complementary activities that do not adversely affect each other. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the immunoglobulin of the invention, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated immunoglobulins remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions. In this regard, reduction/elimination of disulfide forming cysteine residues as described herein may be particularly advantageous.

Uses

An immunoglobulin of the present invention may be used in, for example, in vitro, ex vivo and in vivo therapeutic methods.

For example, the antibodies of the invention can be used as an antagonist to partially or fully block the specific antigen activity in vitro, ex vivo and/or in vivo. Moreover, at least some of the immunoglobulins of the invention can neutralize antigen activity from other species. Accordingly, the antibodies of the invention can be used to inhibit a specific antigen activity, e.g., in a cell culture containing the antigen, in human subjects or in other mammalian subjects having the antigen with which an antibody of the invention cross-reacts (e.g. chimpanzee, baboon, marmoset, cynomolgus and rhesus, pig or mouse). In one embodiment, the immunoglobulin of the invention can be used for inhibiting antigen activities by contacting the immunoglobulin with the antigen such that antigen activity is inhibited. Preferably, the antigen is a human protein molecule.

In another embodiment, an antibody of the invention can be used in a method for inhibiting an antigen in a subject suffering from a disorder in which the antigen activity is detrimental, comprising administering to the subject an immunoglobulin of the invention such that the antigen activity in the subject is inhibited. Preferably, the antigen is a human protein molecule and the subject is a human subject. Alternatively, the subject can be a mammal expressing the antigen with which an antibody of the invention binds. Still further the subject can be a mammal into which the antigen has been introduced (e.g., by administration of the antigen or by expression of an antigen transgene). An immunoglobulin of the invention can be administered to a human subject for therapeutic purposes. Moreover, an immunoglobulin of the invention can be administered to a non-human mammal expressing an antigen with which the immunoglobulin cross-reacts (e.g., a primate, pig or mouse) for veterinary purposes or as an animal model of human disease. Regarding the latter, such animal models may be useful for evaluating the therapeutic efficacy of antibodies of the invention (e.g., testing of dosages and time courses of administration). Blocking antibodies of the invention that are therapeutically useful include, for example but not limited to, anti-VEGF, anti-IgE, anti-CD 11, anti-interferon and anti-tissue factor antibodies. The antibodies of the invention can be used to treat, inhibit, delay progression of, prevent/delay recurrence of, ameliorate, or prevent diseases, disorders or conditions associated with abnormal expression and/or activity of one or more antigen molecules, including but not limited to malignant and benign tumors; non-leukemias and lymphoid malignancies; neuronal, glial, astrocytal, hypothalamic and other glandular, macrophagal, epithelial, stromal and blastocoelic disorders; and inflammatory, angiogenic and immunologic disorders.

In one aspect, a blocking antibody of the invention is specific to a ligand antigen, and inhibits the antigen activity by blocking or interfering with the ligand-receptor interaction involving the ligand antigen, thereby inhibiting the corresponding signal pathway and other molecular or cellular events. The invention also features receptor-specific antibodies which do not necessarily prevent ligand binding but interfere with receptor activation, thereby inhibiting any responses that would normally be initiated by the ligand binding. The invention also encompasses antibodies that either preferably or exclusively bind to ligand-receptor complexes. An antibody of the invention can also act as an agonist of a particular antigen receptor, thereby potentiating, enhancing or activating either all or partial activities of the ligand-mediated receptor activation.

In certain embodiments, an immunoconjugate comprising an antibody conjugated with a cytotoxic agent is administered to the patient. Preferably, the immunoconjugate and/or antigen to which it is bound is/are internalized by the cell, resulting in increased therapeutic efficacy of the immunoconjugate in killing the target cell to which it binds. In one embodiment, the cytotoxic agent targets or interferes with nucleic acid in the target cell. Examples of such cytotoxic agents include any of the chemotherapeutic agents noted herein (such as a maytansinoid or a calicheamicin), a radioactive isotope, or a ribonuclease or a DNA endonuclease.

Antibodies of the present invention can be used either alone or in combination with other compositions in a therapy. For instance, an antibody of the invention may be co-administered with another antibody, chemotherapeutic agent(s) (including cocktails of chemotherapeutic agents), other cytotoxic agent(s), anti-angiogenic agent(s), cytokines, and/or growth inhibitory agent(s). Where an antibody of the invention inhibits tumor growth, it may be particularly desirable to combine it with one or more other therapeutic agent(s) which also inhibits tumor growth. For instance, anti-VEGF antibodies blocking VEGF activities may be combined with anti-ErbB antibodies (e.g. HERCEPTIN® anti-HER2 antibody) in a treatment of metastatic breast cancer. Alternatively, or additionally, the patient may receive combined radiation therapy (e.g. external beam irradiation or therapy with a radioactive labeled agent, such as an antibody). Such combined therapies noted above include combined administration (where the two or more agents are included in the same or separate formulations), and separate administration, in which case, administration of the antibody of the invention can occur prior to, and/or following, administration of the adjunct therapy or therapies.

The antibody of the invention (and adjunct therapeutic agent) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. In addition, the antibody is suitably administered by pulse infusion, particularly with declining doses of the antibody. Preferably the dosing is given by injections, most preferably intravenous or subcutaneous injections, depending in part on whether the administration is brief or chronic.

The antibody composition of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibodies of the invention present in the formulation, the type of disorder or treatment, and other factors discussed above. These are generally used in the same dosages and with administration routes as used hereinbefore or about from 1 to 99% of the heretofore employed dosages.

For the prevention or treatment of disease, the appropriate dosage of an antibody of the invention (when used alone or in combination with other agents such as chemotherapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1 mg/kg-10 mg/kg) of antibody is an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the antibody). An initial higher loading dose, followed by one or more lower doses may be administered. An exemplary dosing regimen comprises administering an initial loading dose of about 4 mg/kg, followed by a weekly maintenance dose of about 2 mg/kg of the antibody. However, other dosage regimens may be useful. The progress of this therapy is easily monitored by conventional techniques and assays.

Articles of Manufacture

In another aspect of the invention, an article of manufacture containing materials useful for the treatment of the disorders described above is provided. The article of manufacture comprises a container and a label or package insert on or associated with the container. Suitable containers include, for example, bottles, vials, syringes, etc. The containers may be formed from a variety of materials such as glass or plastic. The container holds a composition which is by itself or when combined with another compositions effective for treating the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice, such as cancer. Moreover, the article of manufacture may comprise (a) a first container with a composition contained therein, wherein the composition comprises an antibody of the invention; and (b) a second container with a composition contained therein, wherein the composition comprises a further cytotoxic agent. The article of manufacture in this embodiment of the invention may further comprise a package insert indicating that the first and second antibody compositions can be used to treat cancer. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The following are examples of the methods and compositions of the invention. It is understood that various other embodiments may be practiced, given the general description provided above.

The following Examples are provided to illustrate, but not limit, the invention.

### EXAMPLES

### Generation and Characterization of Antibodies Comprising Variant Hinge Regions

For expression and production of wild type and hinge variant antibodies, expression vectors comprising sequences encoding these antibodies are constructed using standard recombinant methods. For example, an expression vector for an antibody can be constructed by inserting a coding sequence for the heavy and light chain of the antibody into a suitable vector backbone. Such vector backbones are numerous and well know in the art, including those described herein. A coding sequence for anti-Tissue Factor (also referred to herein as ATF, anti-TF, and aTF) can be obtained as described in Presta et al., Thromb Haemost. 2001 March; 85(3):379-89. A coding sequence for anti-HER2 can be obtained as described in U.S. Pat. Nos. 5,821,337 and 6,054,297.

Using standard recombinant DNA techniques, expression vectors for production of the anti-TF and anti-HER2 IgG1 antibodies, either in wild type or hinge variant forms, were generated. The antibodies expressed from these vectors were characterized as described below. All vectors comprised an SV40 promoter/enhancer sequence. The anti-TF vectors comprised a DHFR selection marker. The anti-HER2 vectors comprised a DHFR/Puromycin selection marker. Hinge variant sequences were generated by substituting both cysteines in the hinge region with serine, using standard mutagenesis techniques.

DNA of the anti-HER-2 and anti-TF IgG₁ hinge variant constructs was sequenced to confirm the cysteine to serine mutations in the hinge region.

Expression vector DNA was purified and used for transfection of a CHO host cell line (DP12) with lipofectamine (Invitrogen, Calsbad, Calif.) according to manufacturer's instruction. Colonies appearing on plates in the presence of methotrexate were picked and screened for production of antibody in spinners vessels. Production cultures of anti-HER-2 and anti-TF IgG₁ with and without the variant residues were generated. Wild-type and hinge variant cell lines expressing anti-HER-2 and anti-TF IgG1 were cultivated under identical cell culture conditions using standard 1 L spinner vessels. Spinners were seeded at 6×10⁶ cells/ml. Samples were taken daily for determination of cell concentration, viability and titer. Cultures were harvested on day 7 (viability 50-70%). The harvested cell suspension was centrifuged (200 g, 10 min) at low speed, and the cell-free supernatant was filtered (0.2 μm). Antibodies were purified by protein A affinity chromatography (ProSep Protein A) using fast protein liquid chromatography. Proteins were analyzed under non-reducing conditions by SDS PAGE on 4 to 12% gradient gels (NuPAGE) from Invitrogen using MOPS running buffer. Coomassie blue R250 staining solution was used to visualize the protein bands.

Native PAGE Analysis

Purified antibody samples were diluted with Novex® Tris-Glycine Native Sample Buffer (Invitrigen, Calsbad, Calif.) and loaded onto a pre-cast Novex® 16% Tris-Glycine gel. The gel was run in Novex® Tris-Glycine Native running Buffer at 125 volts for 6 to 12 hours. The gel was stained with Coomassie Brilliant blue stain and destained per standard protocols.

MALDI-TOF/MS

Oligosaccharides were released from anti-TF and anti-HER-2 using N-glycosidase F and analyzed by MALDI-TOF/MS as described by Papac, D. I., Briggs, J. B., Chin, E. T., Jones, A. J. S. Glycobiology 8: 445-454 (1998).

FcRn Binding Affinity Measurement by Biacore

A BIAcore-2000 surface plasmon resonance (SPR) system (Biacore Inc., Piscataway, N.J.) was used to determine association (kₒₙ) and dissociation (k_{off}) constants of antibody variants for binding to rat FcRn essentially as previously described (Raghavan, et al., Proc. Natl. Aacad. Sci. USA 92, 11200-11204 (1995); Vaughn & Bjorkman, Biochemistry 36, 9374-9380 (1997); Vaughn et al., J. Mol. Biol. 274, 597-607 (1997)). A CM-5 biosensor chip (Biacore, Inc.) was activated according to the manufacturer's instructions for amine coupling. Rat FcRn was coupled to the chip at a density of about 1400 response units (RU) in 10 mM sodium acetate buffer, pH 4.8. Unreacted groups were blocked with 1 M ethanolamine. The steady-state binding of IgG variants binding to immobilized FcRn was measured with 2-fold serial dilutions beginning with 8 uM IgG at 25° C. in 10 mM Mes-buffered saline pH 6.0, 0.05% Tween-20, 0.01% sodium azide. Regeneration of the chip after each cycle was with phosphate buffered saline pH 7.4, followed by washing, an injection of 10 mM tris-buffered saline pH 8.0, and additional washing.

Plots of the amount of antibody (RU) bound as a function of the concentration of antibody injected were analyzed by fitting a 2-independent-site binding model (Vaughn & Bjorkman, supra) using Kaleidograph software. The fitting equation was as follows:
y=m1*(m2*(x/m3)/(1+(x/m3))+((1−m2)*(x/m4)/(1+x/m4)))
where x is the concentration of antibody, y is the steady-state response (RU), m2 is the fraction of binding sites with a higher apparent affinity (K_{d}¹) given by m3, and m4 is the lower apparent affinity, K_{d}².

The crystal structure of FcRn in complex with IgG (Burmeister et al., Nature 372, 336-343 (1994)) reveals two copies of FcRn bound to one copy of IgG. Formation of the 2:1 complex is likely represented by the high affinity binding sites observed when two copies of FcRn on the biosensor dextran interact with a single copy of IgG (Raghavan et al., supra). The low-affinity interaction (K_{d}²) appeared similar for the two versions; however, greater errors are associated with these measurements.

Prothrombin Time (PT) Assay

Samples of purified anti-TF IgG1 containing either wild type or variant hinge mutated were tested for biological activity in a prothrombin time assay as described in Presta, et. al., Thromb. Haemost. 85: 379-389 (2001). Pooled normal human plasma anticoagulated with sodium citrate (0.38% final) was stored at −70° C. and defrosted in a 37° C. water bath the day of assay. Various concentrations of antibody samples were added to the plasma (dilution made into PBS; 1:10 dilution in plasma) and allowed to incubate at room temperature for 10 minutes. In an IL ACL 6000 coagulometer (Beckman Coulter Inc, Mesa Calif.) 50 μl plasma/antibody sample was mixed with 100 ul Innovin® (Dade Inc, Hialeah Fla.) recombinant human tissue factor/calcium chloride PT reagent. Time to clot formation, as detected optically, was measured. Results were expressed as fold prolongation of PT over mean control sample clotting times (plasma+PBS only). A 4-parameter curve (KaleidaGraph, Synergy Software, Reading Pa.) was fit to the dose-response data by the equation ((m1−m4)/(1+(m0/m3)ˆm2))+m4 where m1=the maximal clotting time, m2=the slope of the curve, m3=the inflection point of the curve, and m4=the minimal clotting time. The concentration of each sample which prolonged the clotting time two-fold was calculated from this curve by the equation x=m3(((m1−m4)/(2−m4))−1)ˆ(1/m2).

Pharmacokinetics Study of Anti-TF IgG1

Two groups of 4 Sprague Dawley (SD) rats each were administered a single IV bolus dose (3 mg/kg) of anti-TF IgG1 or hinge variant anti-TF IgG1. Plasma samples were collected out to 42 days for analysis by TF-binding assay. Pharmacokinetic parameter estimates were determined using a 2-compartment elimination model in WinNonlin 3.0. The following PK parameters were estimated: Clearance (CL), Volume of distribution (V1), maximum plasma concentration (Cmax), drug exposure as measured by the area under the concentration versus time curve (AUC), Steady State Volume (Vss), alpha half-life (a-HL), and beta half-life (b-HL). Statistical comparisons between groups were done by an ANOVA.

Complement C1q Binding

Binding of antibody to C1q was evaluated by a method modified from that previously described by Idusogie E. E. et al., J. Immunol. 164: 4178-4184 (2000). Serial dilutions of the hinge variant and control antibodies were coated onto the assay plates in carbonate buffer (50 mM, pH 9.6) overnight at 4° C. The plates were blocked and washed with 0.5% BSA in PBS and subsequently incubated with 0.05% Tween-20 in PBS. After coating, the plates were incubated with purified human C1q in assay buffer (0.5% BSA, 0.05% Tween-20, 0.05% ProClin 300 in PBS) for 2 hours. The bound C1q was detected with goat anti-human C1q followed by horseradish peroxidase-conjugated donkey anti-goat IgG. The plates were developed with tetramethylbenzidine as substrate and EC₅₀ values for binding of the antibodies to C1q were determined.

Cell Lysis Via ADCC with Peripheral Blood Mononuclear Cells

ADCC was assessed using peripheral blood mononuclear cells (PBMCs) from healthy donors as effector cells. Briefly, buffy coats were obtained from Stanford Blood Bank, heparinized fresh blood obtained from Genentech normal donor, and PBMCs were isolated by ficoll gradient centrifugation. PBMCs were then cultured in RPMI-1640 with 10% fetal bovine serum at 37° C. and 5% CO2 for 18-22 hours before use in the assay. Target cells were seeded into each well of a 96-well round bottom plate. Serial dilutions of test antibody were added to the cells to allow opsonization. After 45 minutes at 37° C. and 5% CO2, PBMCs were added for an effector:target ratio of 30:1, and the plates were further incubated. At the end of incubation, plates were centrifuged. Supernatants were transferred to corresponding wells of an optically clear 96-well flat bottom plate, and the levels of LDH released were measured. Absorbance of wells containing intact target cells was set as low control. Complete lysis was achieved by addition of 2% Triton X-100 (high control). Antibody-independent cellular cytotoxicity (AICC) was measured through mixing target and effector cells in the absence of test antibody. The specific % cytotoxicity was calculated as follows:

$$\% Cytotoxicity = 100 \times \frac{A 490 nm Sample - A 490 nm AICC OD}{A 490 nm High Control - A 490 nm Low Control}$$

The absorbance values were plotted against the antibody concentration, and the EC50 values were generated by fitting the data to a 4-parameter equation with SoftMax Pro (Molecular Devices, Sunnyvale, Calif.).

Assessment of Complement Dependent Cytotoxicity (CDC) Activity

CDC activity is mediated through the C1q component of the complement. An anti-CD20 antibody comprising either wild type or variant (i.e., cysteines converted to serines) hinge regions was analyzed. The wild type form of this antibody was previously shown to have CDC activity.

Binding to Human Fc Gamma Receptors

Binding of antibody to the human Fc gamma receptors (FcγR) was assessed by modifications of procedures described by Shields R. L. et al., J. Biol. Chem. 276(9): 6591-6604 (2001). Monomeric IgG is capable of binding to the high affinity FcγRIa (CD64); however, the low affinity receptors, FcγRIIa (CD32A), FcγRIIb (CD32B), and FcγRIIIa (CD16) require multimeric IgG for binding. Therefore, for the low affinity receptor binding assays, dimers of the antibodies were formed by mixing antibody with goat anti-human kappa chain at a molar ratio of 2:1. The FcγR were expressed as recombinant fusion proteins of the extracellular domain of the receptor alpha chains with Gly/His₆/GST. Anti-GST-coated, BSA-blocked assay plates were used to capture the FcγR. The plates were washed after this and all subsequently incubated with 0.05% Tween-20 in PBS. The receptors were incubated for 2 hours with serial dilutions of hinge variant and control (wild type counterpart) antibodies as monomers for FcγRIa and as multimers for the low affinity FcγR. The bound antibody was detected with horseradish peroxidase-conjugated goat anti-human F(ab′)2. The plates were developed with tetramethylbenzidine as substrate and EC₅₀ values for binding of the antibodies to the FcγR were determined.

Xenograft Study

The anti-HER-2 variants were tested against the MMTV-HER2 F2#1282 mammary tumor transplants in beige nude mice. MMTV-HER2 F2#1282 mammary tumor was from a HER2 transgenic mouse whose HER2 expression is targeted to the mammary gland using the MMTV promoter. See U.S. Pat. Application Nos. 20020001587 and 20020035736.

The tumors overexpressed HER2 and were maintained in vivo as a transplanted tumor line. For this study, the tumors were surgically transplanted as ˜2 mm×2 mm chunks of tissue into the right #2,3 mammary fat pad of wild type beige nude mice. 14 days after the transplant, the study began with mean tumor volumes between 150 to 200 mm³ (individual tumor sizes ranged from 70 to 400 mm³).

4 mice per group were used and anti-HER-2 administered:

Group A: Herceptin (commercially available anti-HER-2; Genentech, Inc., South San Francisco) 10 mg/kg IP once per week for 4 weeks

Group B: Herceptin 30 mg/kg IP, once per week for 4 weeks

Group C: anti-HER-2 hinge variant 30 mg/kg IP once per week for 1 week

(Commercially available Herceptin was used in Groups A and B.)

Results and Discussion

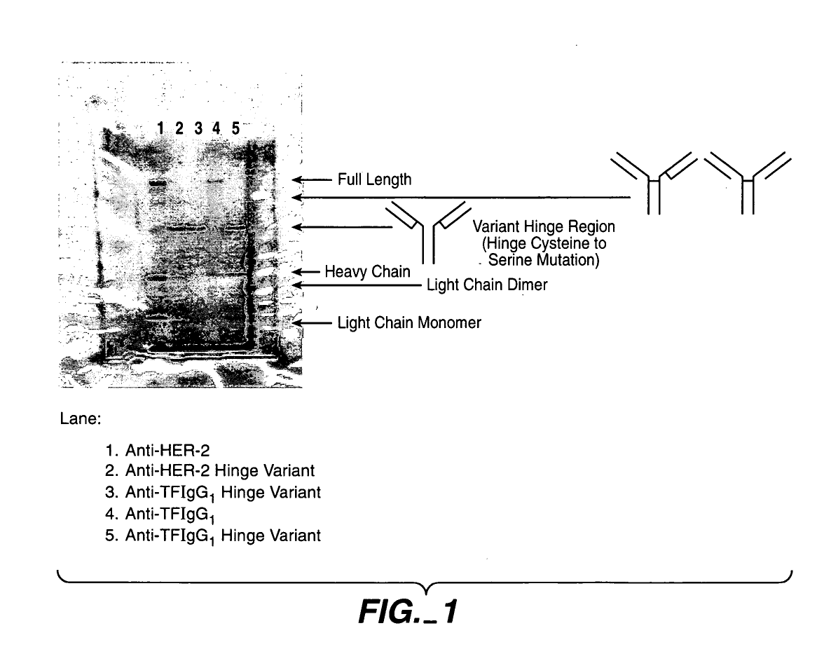

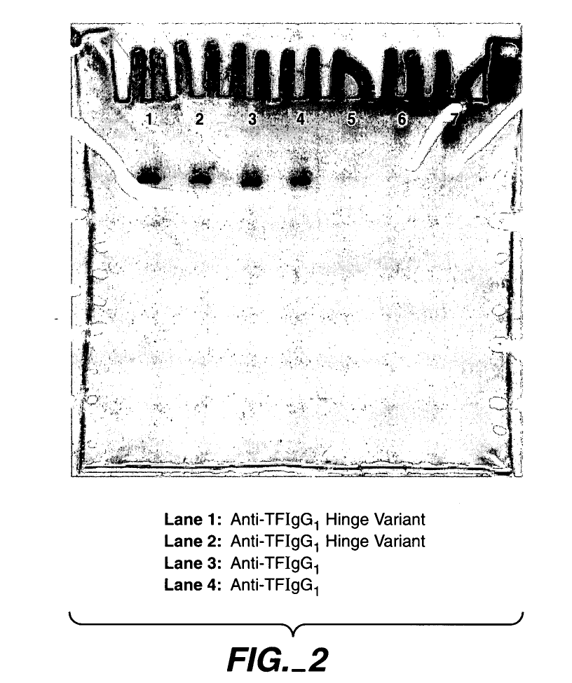

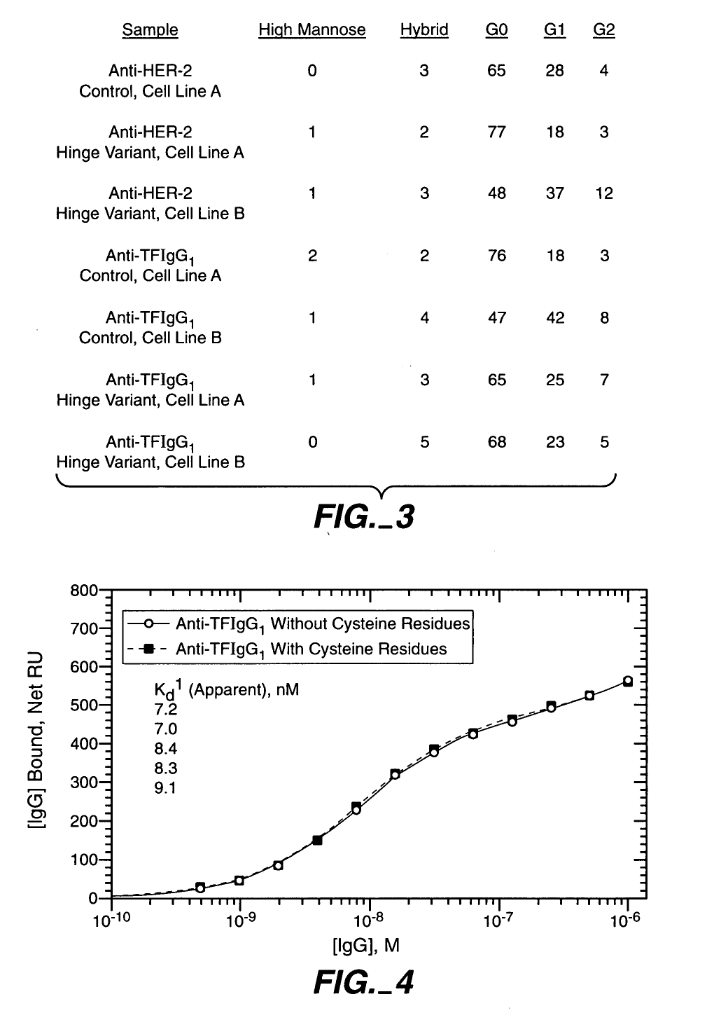

Purified samples from spinner productions were run on a SDS PAGE gel to confirm the expression of antibody without disulfide bonds (FIG. 1). For anti-HER-2 and anti-TF IgG₁ without the cysteine residues, a predominant band could be observed at about 75 kD consistent with the presence of the heavy-light chain antibody form that lacks the interchain disulfide bonds at this molecular weight. Native PAGE gel analysis (FIG. 2) showed that anti-TF IgG1 molecules with the mutated hinge region stayed associated. This suggests that non-covalent interactions are sufficient to hold the dimer together. MALDI/TOF-MS analysis (FIG. 3) confirmed that the removal of disulfide bonds had no impact on the two N-linked oligosaccharides in the Fc region. Glycosylation patterns looked similar to antibodies without the mutation. This series of assays confirmed that antibodies containing the hinge cysteine mutations posses the same physical/analytical properties as their wild type counterparts.

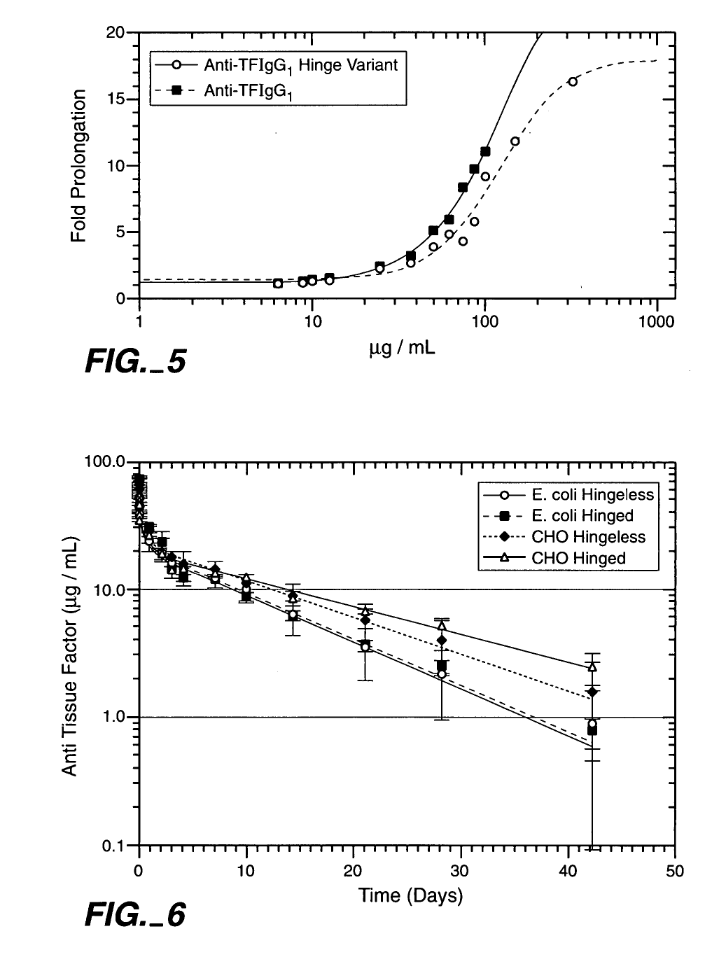

Anti-TF IgG₁ comprising either the hinge cysteines or the hinge cysteines mutated to serines were evaluated in the prothrombin time assay. As shown in FIG. 5, both versions of the molecule showed no statistically significant difference in fold prolongation of PT (prothrombin time). Additionally, the two antibodies were also tested in a Biacore FcRn binding assay (FIG. 4). Anti-TF IgG₁ with the cysteine to serine mutation showed equivalent FcRn binding as its wild type counterpart. Moreover, a pharmacokinetics study that was done in rats showed identical clearance properties in vivo (FIG. 6). Anti-TF IgG₁ without the cysteines in the hinge region showed no statistically significant difference in clearance compared to antibody with the cysteines (anti-TF IgG₁ with cysteine residues 8.24±0.55 ml/day/kg and anti-TF IgG₁ without cysteine residues 10.47±2.62 ml/day/kg). The data demonstrated that FcRn binding, pharmacokinetic properties as well as clot formation of the anti-TF IgG₁ molecule are not substantially affected by the mutation in the hinge region, in comparison to levels observed in wild type forms of the molecule.

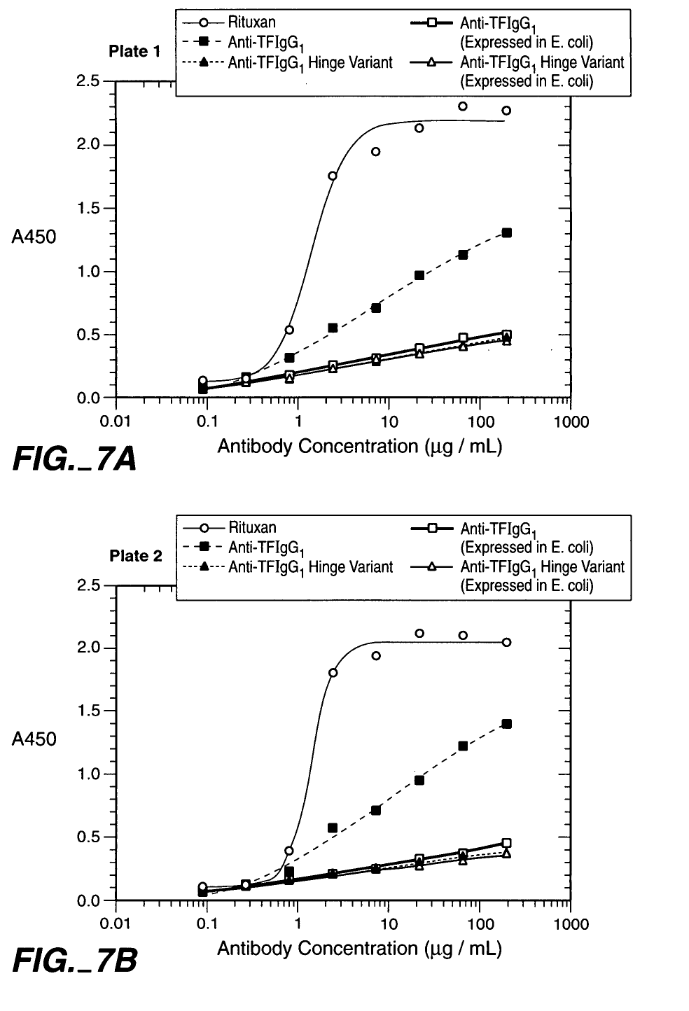

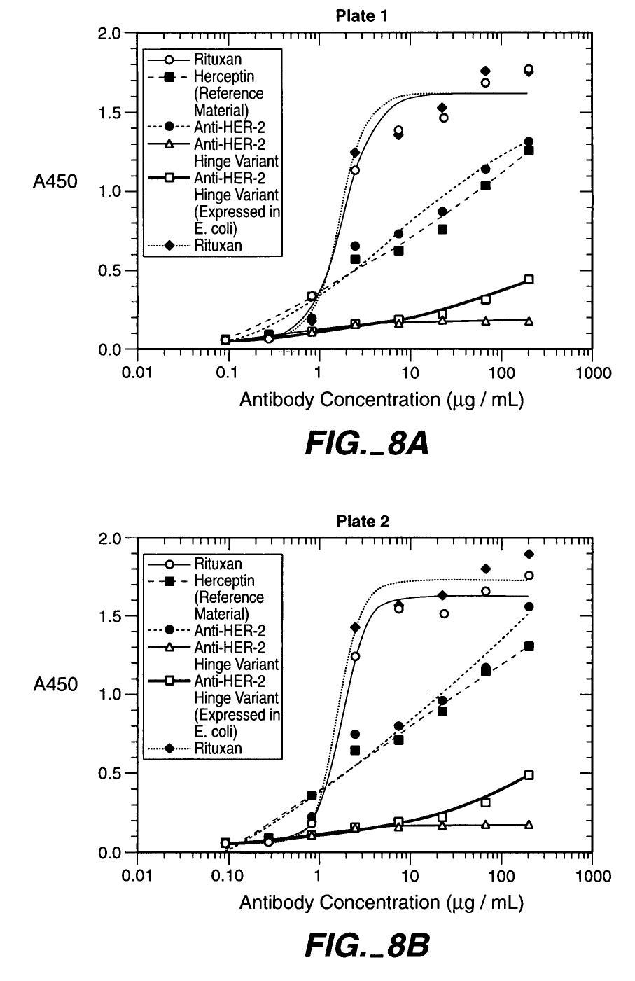

Anti-HER-2 and anti-TF IgG1 with and without the cysteine to serine mutation in the hinge region were evaluated in a complement C1q binding assay. Complement activation occurs by binding of C1q to the Fc domain of IgGs. As shown in FIG. 7 and FIG. 8, anti-HER-2 and anti-TF IgG₁ comprising hinge variant regions expressed in mammalian cells showed a significant decrease in complement C1q binding. In the case of an anti-CD20 antibody, for which CDC activity was assessed for both wild type and hinge variant forms, C1q binding was significantly reduced for hinge variant antibody in comparison to the wild type counterpart. For example, EC 50 of the hinge variant was 1.14 μg/ml compared to 0.62 μg/ml for the control material. Although the hinge variant antibody still showed some binding to C1q, the binding was apparently not sufficient to mediate a CDC response. In a CDC assay using WIL-2 cells as target cells and PBMC cells as effector cells, no activity could be measured for the hinge variant antibody (data not shown).

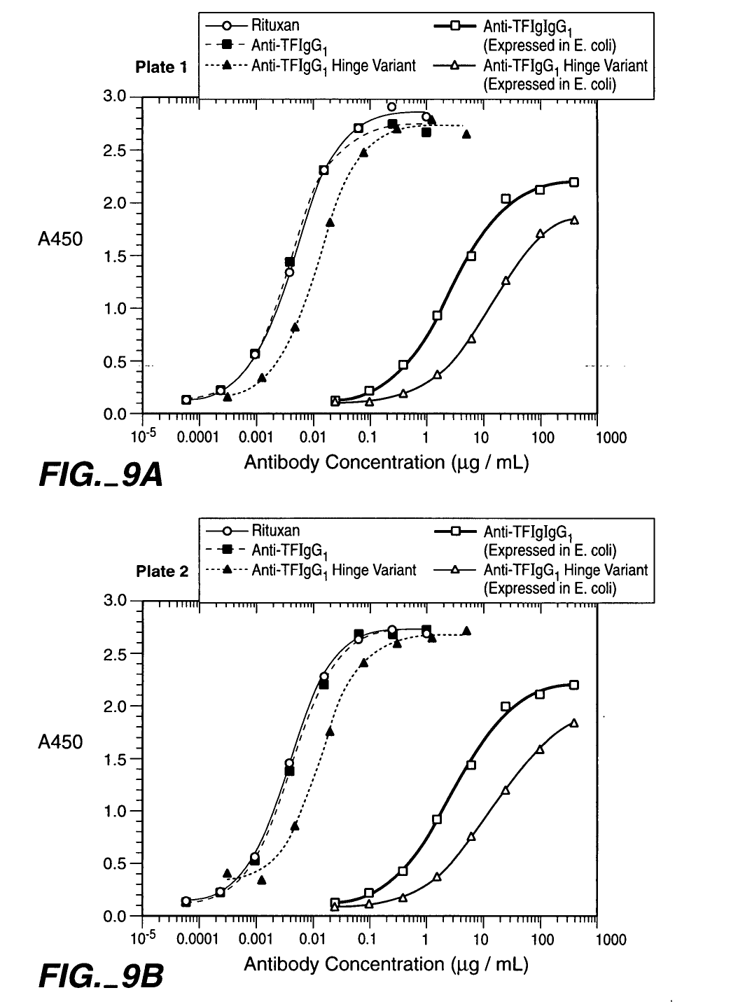

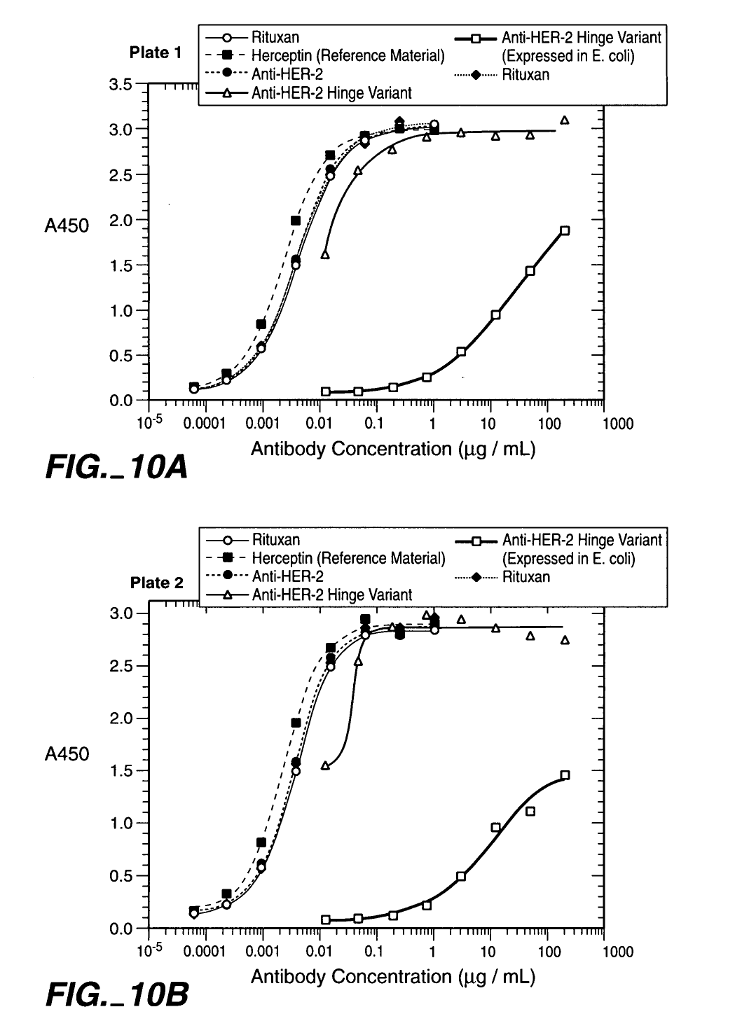

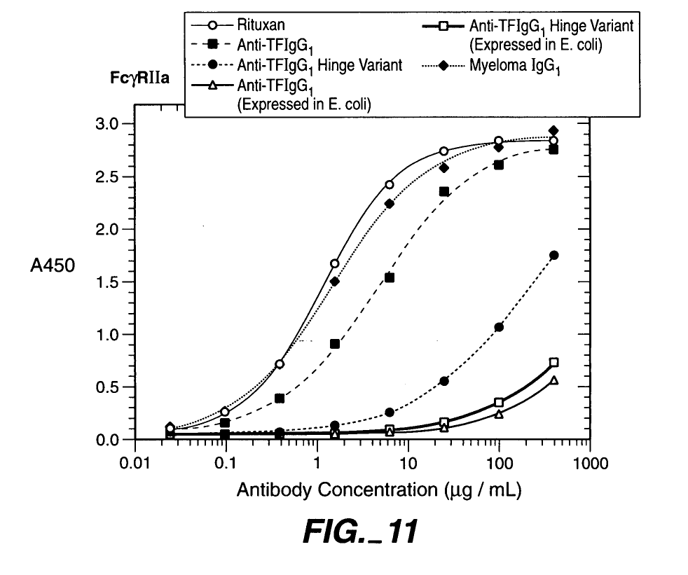

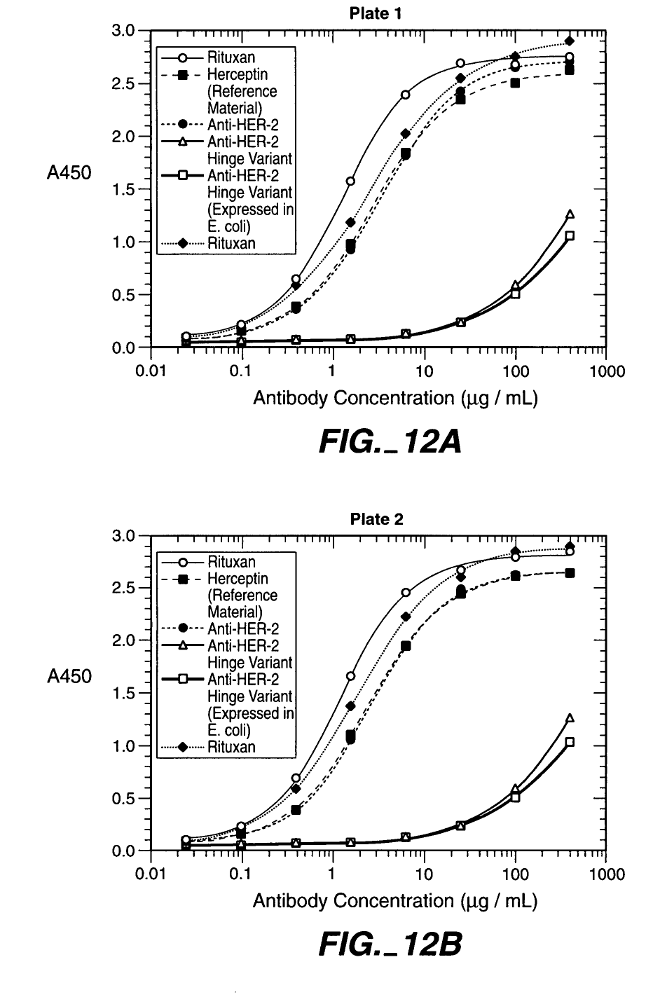

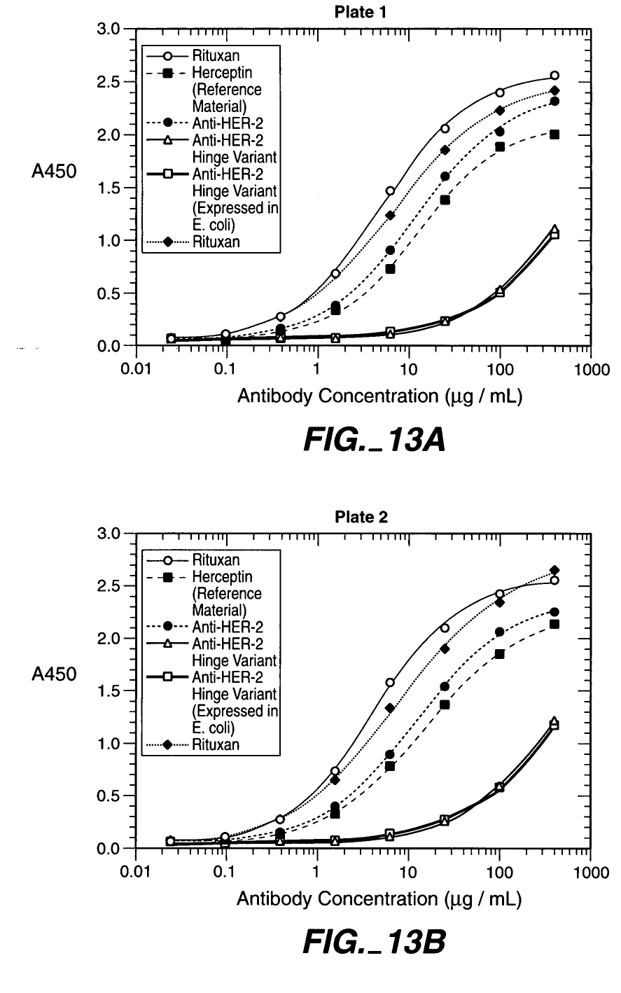

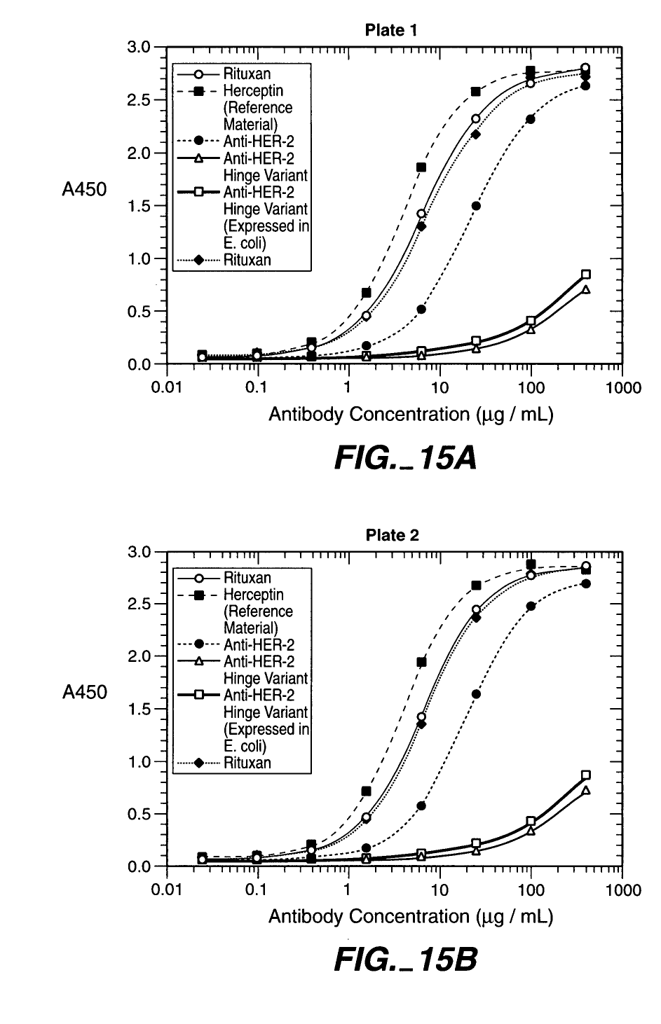

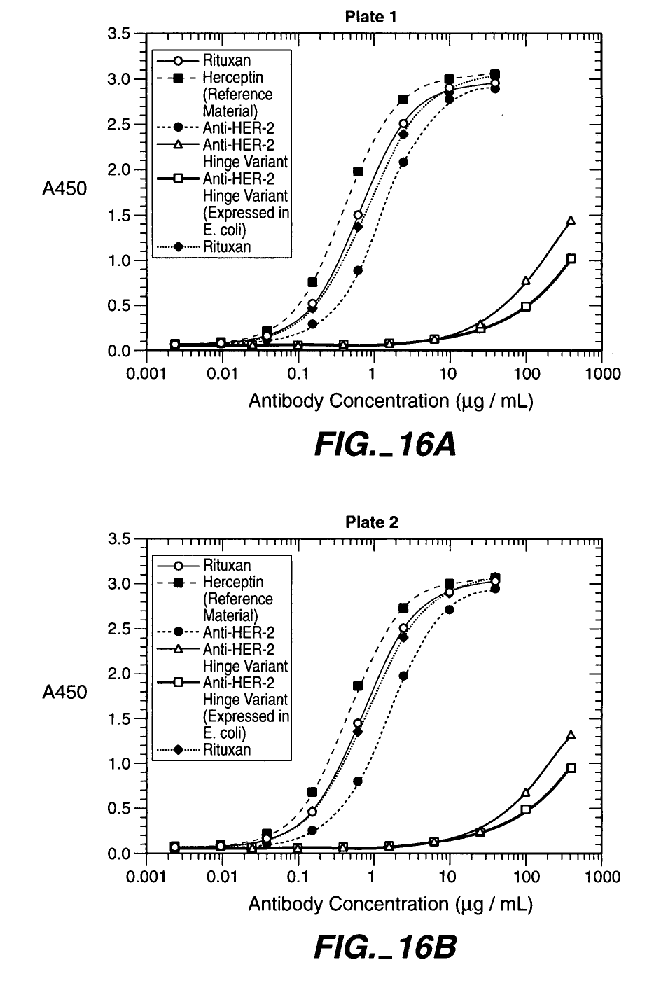

Both the anti-HER2 and anti-TF variant antibodies were tested in a panel of Fcγ receptor binding assays. For the specific recognition of Fc receptors only one chain of the receptor is required, and the γ chain mediates the signal transduction. Commercially available Herceptin and Rituxan as well as purified material from wild-type anti-HER-2 and anti-TF IgG1 cell lines were used as control cases. As expected, dramatically reduced or essentially no binding (compared to control material) could be observed for full length anti-HER-2 and anti-TF IgG1 expressed in E. coli in all effector function assays performed (FIG. 7-16). Hinge variant anti-HER-2 and anti-TF IgG1 expressed in CHO cells showed a small decrease in binding the FcγIa receptor compared to their wild type counterparts (FIGS. 9 and 10). In addition, both hinge variants exhibited a significant reduction, at levels similar to the material produced in E. coli, in binding the FcγIIa and FcγIIb receptors (FIGS. 11, 12, 13).

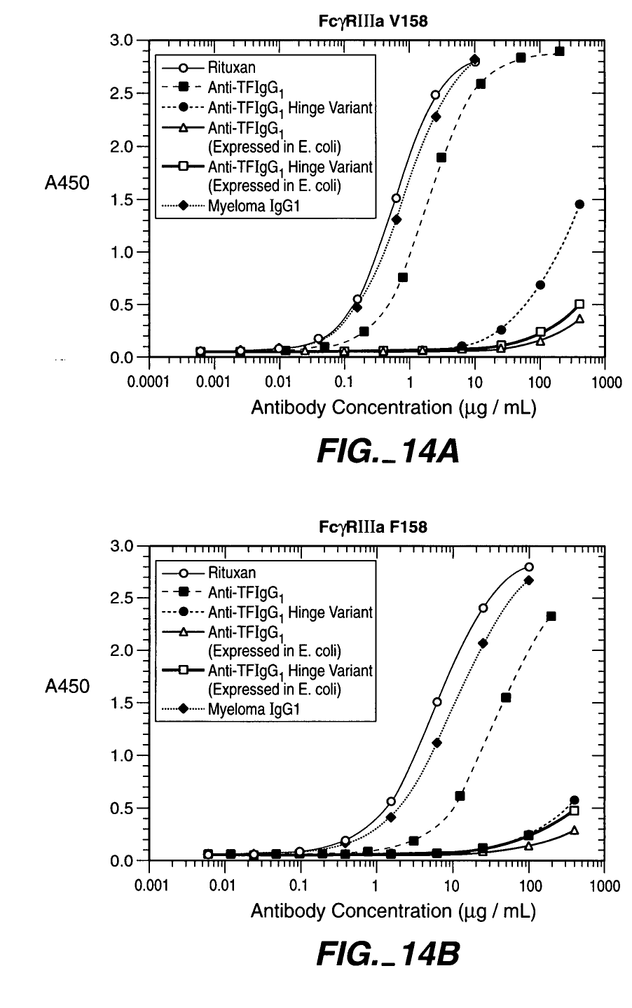

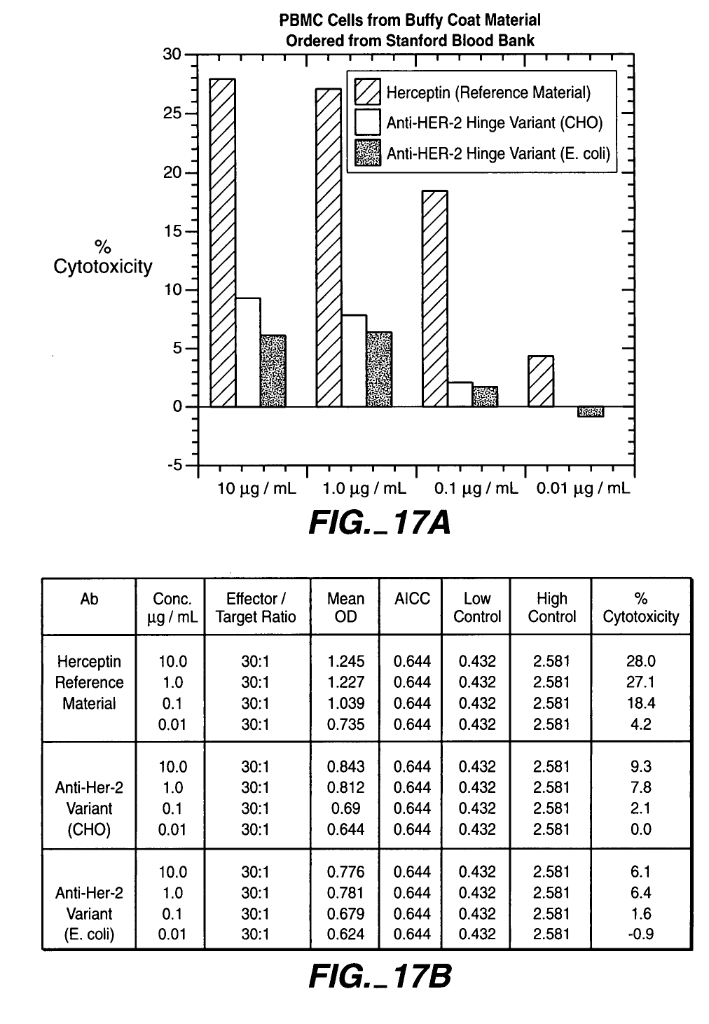

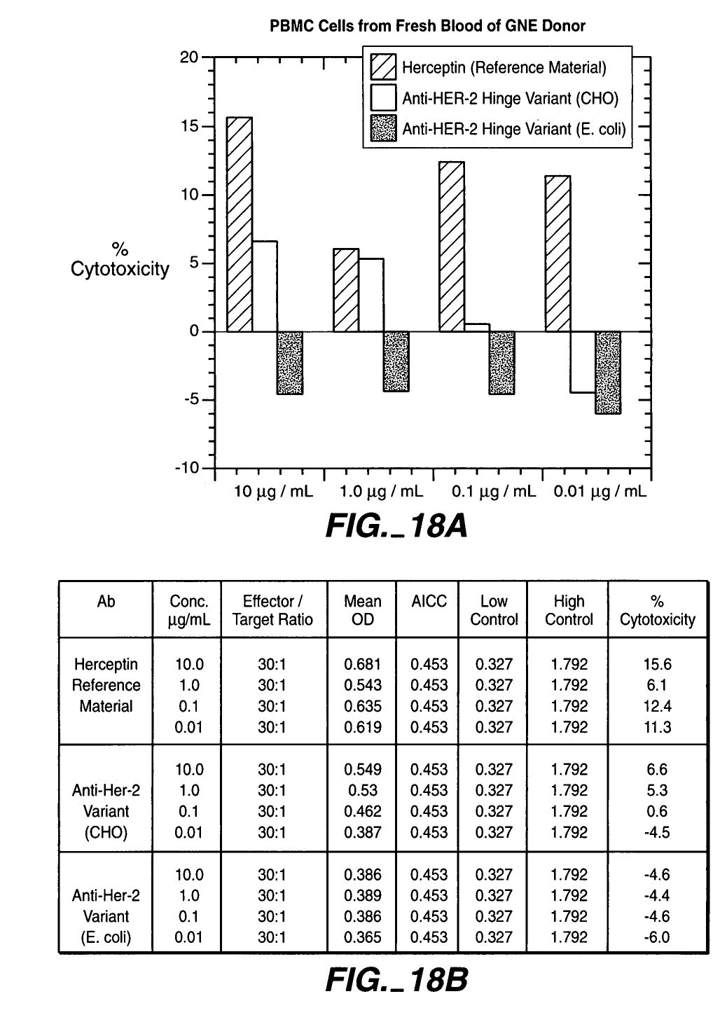

Hinge variant antibodies (e.g., anti-HER-2) and their wild type counterparts were also evaluated in FcγRIII binding and ADCC activity assays. Anti-HER-2 and anti-TF IgG1 molecules lacking the disulfide bonds in the hinge region showed a dramatic decrease in FcγRIII binding compared to material without the deletion. The decline in binding could be observed for the high affinity allele V158 as well as for the low affinity allele F158 (FIGS. 14, 15, 16) and was similar to the binding capacity of material produced in E. coli. Since the FcγIII receptor is the primary receptor responsible for ADCC, anti-HER-2 and hinge variant anti-HER-2 were also tested in two independent ADCC assays using PBMC cells as effector cells and SKBR3 cells as target cells (FIGS. 17 and 18). In both assays, cytotoxicity of anti-HER-2 without the cysteine residues in the hinge region was substantially reduced compared to reference material. Interestingly, the level of cytotoxicity of hinge variant anti-HER-2 expressed in either CHO or E. coli cells was apparently in part dependent on the donor that was used to isolate effector cells. Using fresh donor blood, ADCC activity of variant antibodies was more reduced than in similar assays using frozen donor samples when compared to wild type material; for E. coli, ADCC activity was not even present (FIG. 18). Nonetheless, in all cases examined, the levels of cytoxicity of hinge variant antibodies were significantly lower than those observed in wild type counterparts assessed under similar assay conditions.

For illustrative purposes, a summary of numerical values obtained for Fcγ receptor and C1q binding is provided in Tables 2-3.

TABLE 2
Fcγ Receptor and C1q Binding ELISAs: Summary
Trial 1

| | EC50 (μg/mL) | | | | | | |
|---|---|---|---|---|---|---|---|
| | | | | | | C1q* | |
| Sample | RIa | RIIa | RIIb | RIIIa(F158) | RIIla(V158) | Plate 3 | Plate 4 |
| Rituxan (Positive Ctrl) | 0.0039 | 1.2 | 5.1 | 6.2 | 0.59 | 1.4/100 | 1.4/100 |
| ATF CHO Wild type | 0.0037 | 4.4 | 22.5 | 39.00 | 1.9 | —/58 | —/66 |
| ATF Variant | ˜0.01 | ˜200 | ˜300 | >400 | ˜400 | —/18 | —/14 |
| ATF E coli Wild type | 3.8 | >400 | >400 | >400 | >400 | —/19 | —/18 |
| ATF E coli Variant | 16.6 | >400 | >400 | >400 | >400 | —/16 | —/14 |

Trial 2 for FcγRIa

EC50 (μg/mL)

| Sample | Plate 1 | Plate 2 |
|---|---|---|
| Rituxan | 0.0039 | 0.0046 |
| ATF CHO wild type | 0.0042 | 0.0039 |
| ATF CHO Variant | 0.013 | 0.011 |
| ATF E coli Wild type | 2.8 | 2.6 |
| ATF E coli Variant | 14.6 | 13.1 |

*EC50 value (μg/mL)/% Maximal Binding (relative to Rituxan control)

TABLE 3
Fcγ Receptor and C1q Binding ELISAs: Summary
Trial 1

| | EC50 (μg/mL) | | | | | | | | | | | |
|---|---|---|---|---|---|---|---|---|---|---|---|---|
| | RIa | | RIIa | | RIIb | | RIIIa(F158) | | RIIIa(V158) | | C1q* | |
| Sample | Plate 1 | Plate 2 | Plate 3 | Plate 4 | Plate 5 | Plate 6 | Plate 7 | Plate 8 | Plate 9 | Plate 10 | Plate 1 | Plate 2 |
| Rituxan (Positive | 0.0038 | 0.0043 | 1.3 | 1.2 | 5.1 | 4.2 | 6.6 | 6.3 | 0.63 | 0.72 | 1.8 | 1.8 |
| control) | | | | | | | | | | | | |
| Herceptin | 0.002 | 0.002 | 2.8 | 2.3 | 15.4 | 15.4 | 3.90 | 3.70 | 0.40 | 0.45 | ˜25 | ˜20 |
| Anti-HER2 wild type | 0.0035 | 0.0039 | 3.2 | 2.5 | 12.2 | 12.2 | 22.2 | 19 | 1.3 | 1.5 | ˜15 | ˜15 |
| Anti-HER2 variant | ˜0.01 | ˜0.01 | ˜400 | ˜400 | >400 | >400 | >400 | >400 | ˜400 | ˜400 | >400 | >400 |
| E. coli variant | ˜200 | ˜50 | >400 | >400 | >400 | >400 | >400 | >400 | >400 | >400 | >400 | >400 |
| Rituxan (Control) | 0.0037 | 0.0041 | 2.4 | 1.8 | 7.9 | 7.9 | 7.6 | 7.2 | 0.81 | 0.82 | 1.7 | 1.7 |

Trial 2 for FcγRIa

EC50 (μg/mL)
RIa

| Sample | Plate 1 | Plate 2 |
|---|---|---|
| Rituxan | 0.0038 | 0.0046 |
| Anti-HER2 wild type | 0.0038 | 0.0041 |
| Anti-HER2 variant | 0.010 | 0.0090 |
| E. coli variant | 17.0 | 17.8 |

*Note:
curves appear to be plateauing at a lower level of maximum binding

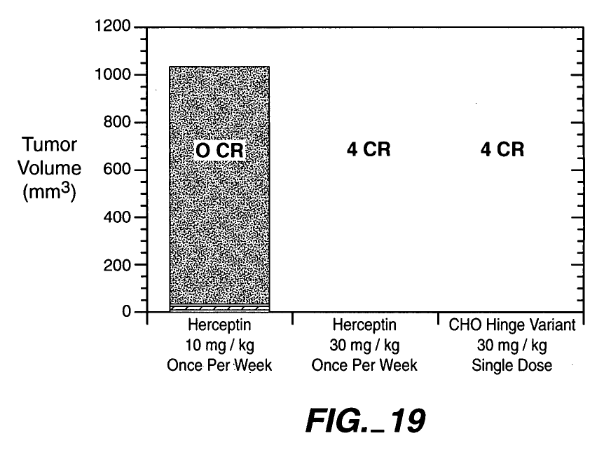

In vivo properties of the anti-HER-2 wild type (Herceptin) and hinge variant were tested in a xenograft model that does not depend on effector functions. MMTV-HER2 F2#1282 mammary tumor transplants were used in beige nude mice. FIG. 19 shows the mean tumor volume of mammary tumor transplants in beige nude mice after exposure to anti-HER-2. A complete response was observed for both wild type anti-HER-2 (Herceptin) and hinge variant anti-HER-2 in all mice treated with the higher dose (30 mg/kg once per week for one week). Thus, it appeared that the therapeutic efficacy of the hinge variant antibody is substantially similar compared to a wild type counterpart which has been shown clinically to be an efficacious therapeutic agent.

1. A method of treating a disease comprising administering to a subject having the disease an antibody effective in treating the disease, wherein said antibody comprises a variant heavy chain hinge region incapable of inter-heavy chain disulfide linkage, and wherein the antibody is produced in a eukaryotic host cell culture.

2. The method of claim 1, wherein the antibody has reduced antibody-dependent cellular cytotoxicity (ADCC) compared to a wild type antibody.

3. The method of claim 1, wherein said variant heavy chain hinge region lacks a cysteine residue capable of forming a disulfide linkage.

4. The method of claim 3, wherein said disulfide linkage is intermolecular.

5. The method of claim 4, wherein said intermolecular disulfide linkage is between cysteines of two immunoglobulin heavy chains.

6. The method of claim 2, wherein a hinge region cysteine residue that is normally capable of forming a disulfide linkage is deleted.

7. The method of claim 2, wherein a hinge region cysteine residue that is normally capable of forming a disulfide linkage is substituted with another amino acid.

8. The method of claim 7, wherein said cysteine residue is substituted with serine.

9. The method of claim 1, wherein the antibody is a full-length antibody.

10. The method of claim 9, wherein said full-length antibody comprises a heavy chain and a light chain.

11. The method of claim 1, wherein said antibody is humanized.

12. The method of claim 1, wherein said antibody is human.

13. The method of claim 1, wherein said antibody is an antibody fragment.

14. The method of claim 13 wherein said antibody fragment is an Fc fusion polypeptide.

15. The method of claim 1, wherein said antibody comprises a heavy chain constant domain and a light chain constant domain.

16. The method of claim 1, wherein the antibody is of an isotype selected from the group consisting of IgG, IgA and IgD.

17. The method of claim 16, wherein the antibody is an IgG.

18. The method of claim 17, wherein the antibody is an IgG1.

19. The method of claim 16, wherein the antibody is an IgG2.

20. The method of claim 1, wherein the antibody is a therapeutic antibody.

21. The method of claim 1, wherein the antibody is an agonist antibody.

22. The method of claim 1, wherein the antibody is an antagonistic antibody.

23. The method of claim 1, wherein the antibody is a diagnostic antibody.

24. The method of claim 1, wherein the antibody is a blocking antibody.

25. The method of claim 1, wherein the antibody is a neutralizing antibody.

26. The method of claim 1, wherein the antibody is capable of binding to a tumor antigen.

27-40. (canceled)

41. The method of claim 1 wherein the antibody is conjugated with a heterologous moiety.

42. The method of claim 41, wherein said heterologous moiety is a cytotoxic agent.

43. The method of claim 42, wherein said cytotoxic agent is selected from the group consisting of a radioactive isotope, a chemotherapeutic agent and a toxin.

44. The method of claim 43, wherein the toxin is selected from the group consisting of calichemicin, maytansine and trichothene.

45-46. (canceled)

47. The method of claim 1, wherein the antibody exhibits substantially similar pharmacokinetic values as its wild type counterpart which comprises wild type ADCC activity.

48. The method of claim 1, wherein the ADCC activity is measured in vitro.

49. The method of claim 1, wherein the eukaryotic host cell is a mammalian host cell.

50. The method of claim 49, wherein the host cell is a Chinese hamster ovary (CHO) cell.

51. The method of claim 1, wherein the antibody has substantially reduced complement dependent cytotoxicity compared to its wild type counterpart antibody.

52. The method of claim 1, wherein the antibody comprises substantially reduced binding to a complement protein compared to its wild type counterpart antibody.

53. The method of claim 52, wherein the complement is C1q.

54. The method of claim 1, wherein the disease is a tumor or cancer.

55. The method of claim 1, wherein the disease is an immunological discorder.

56. The method of claim 55, wherein the immunological disorder is autoimmune.

57-60. (canceled)
